SEQUENCE LISTING (7 sequences)

SEQ ID NO: 1 - DNA, 20 bp, artificial
  Primer sequence for GAPDH
ctggagaaacctgccaagta

SEQ ID NO: 2 - DNA, 20 bp, artificial
  Primer sequence for GAPDH
agtgggagttgctgttgaag

SEQ ID NO: 3 - DNA, 20 bp, artificial
  Primer sequence for Arc
tgagaccagttccactgatg

SEQ ID NO: 4 - DNA, 20 bp, artificial
  Primer sequence for Arc
ctccagggtctccctagtcc

SEQ ID NO: 5 - protein, 5 aa, artificial
  homer binding motif
PPXXF

SEQ ID NO: 6 - protein, 7 aa, artificial
  Homer binding motif
LPSSPSS

SEQ ID NO: 7 - protein, 16 aa, Artificial
  Consensus sequence for eEF2K phosphorylation
RKKYKFNEDTERRRFL

(19) United States
(12) Patent Application Publication
(10) Pub. No.: US 2011/0183942 A1
(43) Pub. Date: Jul. 28, 2011

(54) Methods and Compositions for Treating Alzheimer's Disease

(75) Inventors: Paul Worley, Baltimore, MD (US); Sungjin Park, Baltimore, MD (US); Alexey G. Ryazanov, Princeton, NJ (US)

(73) Assignees: UNIVERSITY OF MEDICINE AND DENTISTRY OF NEW JERSEY, Somerset, NJ (US); JOHNS HOPKINS UNIVERSITY, Baltimore, MD (US)

(21) Appl. No.: 13/054,203

(22) PCT Filed: Jul. 15, 2009
(86) PCT No.: PCT/US09/50767

§ 371 Date: Apr. 4, 2011

Related U.S. Application Data
(60) Provisional application No. 61/135,073, filed on Jul. 15, 2008.

(51) Int. Cl. A61K 31/661; A61K 31/519; A61K 31/4164; A61K 31/445; A61K 31/221; A61K 31/55; A61K 31/13; A61K 31/428; A61P 25/28

(52) U.S. Cl. 514/114; 514/267; 514/396; 514/319; 514/551; 514/215; 514/662; 514/367; 514/297

(57) ABSTRACT

A method is disclosed for inhibiting the build-up of amyloid plaques in the brain of a patient with at least one risk factor for, or a diagnosis of, Alzheimer's Disease by administering to the patient an amount of one or more compounds effective to inhibit the phosphorylative activity of eEF2K, thereby inhibiting amyloid plaque deposition.

### CROSS-REFERENCE TO RELATED APPLICATION

The instant application claims 35 U.S.C. §119(e) priority to U.S. Provisional Patent Application Ser. No. 61/135,073 filed Jul. 15, 2008, the disclosure of which is incorporated by reference.

### FIELD OF THE INVENTION

The present invention relates to methods for inhibiting the development of amyloid plaque deposits in a patient with risk factors for or a diagnosis of Alzheimer's Disease a by administering to the patient a therapeutically effective amount of one or more compounds that inhibit the phosphorylative activity of eEF2K.

### BACKGROUND OF THE INVENTION

Alzheimer's Disease (AD) is a progressive neurodegenerative disorder marked by loss of memory, cognition, and behavioral stability. AD afflicts 6-10% of the population over age 65 and up to 50% over age 85. It is the leading cause of dementia and the third leading cause of death after cardiovascular disease and cancer. There is currently no effective treatment for AD. The total net cost related to AD in the U.S. exceeds $100 billion annually.

While methods of treatment are desirable, AD does not have a simple etiology. It is associated with certain risk factors including (1) age, (2) family history (3) genetics, and (4) head trauma with other epidemiological factors including environmental toxins and low level of education. Specific neuropathological lesions in the limbic and cerebral cortices include intracellular neurofibrillary tangles consisting of hyperphosphorylated tau protein and the extracellular deposition of fibrillar aggregates of amyloid beta peptides (amyloid plaques). The major component of amyloid plaques are the amyloid beta peptides of various lengths. A variant thereof, which is the Aβ 1-42-peptide (Aβ-42), is believed to be the major causative agent for amyloid formation. Another variant is the Aβ 1-40-peptide (Aβ-40). Amyloid beta is the proteolytic product of a precursor protein, beta amyloid precursor protein (beta-APP or APP).

Familial, early onset autosomal dominant forms of AD have been linked to missense mutations in the β-amyloid precursor protein (β-APP or APP) and in the presenilin proteins 1 and 2. In some patients, late onset forms of AD have been correlated with a specific allele of the apolipoprotein E (ApoE) gene, and, more recently, the fording of a mutation in α2-macroglobulin, which may be linked to at least 30% of the AD population. Despite this heterogeneity, all forms of AD exhibit similar pathological findings. Genetic analysis has provided the best clues for a logical therapeutic approach to AD. All mutations, found to date, affect the quantitative or qualitative production of the amyloido-genic peptides known as Aβ-peptides, specifically Aβ-42, and have given strong support to the “amyloid cascade hypothesis” of AD. The likely link between Aβ peptide generation and AD pathology emphasizes the need for a better understanding of the mechanisms of Aβ production and strongly warrants a therapeutic approach at modulating Aβ levels.

Several approaches are presently being pursued to prevent, inhibit, and/or treat AD, including the development of compounds that target enzymes that, in some respect, catalyze Aβ peptide generation and plaque formation. The enzyme Elongation Factor 2 Kinase (eEF2K) presents potential as one such target. eEF2K belongs to a novel family of protein kinases, with prototypical member being Dictyostelium myosin heavy chain kinase A (MHCK A), which display no homology to conventional eukaryotic protein kinases. This protein kinase is highly specific to eEF2 and is responsible for eEF2 phosphorylation. eEF2 promotes ribosomal translocation, the reaction that results in the movement of the ribosome along mRNA during translation. eEF2 was identified among the most prominently phosphorylated proteins in crude tissue and cell lysates. Importantly, it was found that phosphorylation of eEF2 arrests translation, suggesting that this may be a critical mechanism by which the rate of protein synthesis is regulated (Ryazanov et al., FEBS Lett., 214, 331-334 (1987)). This enzyme was previously shown to have increased activity in human brains of individuals with AD (Li, et al., FEBS J., 272, 4211-4220 (2005)) although the mechanism and relevance of the enzyme for such purposes was not clear. Moreover, the relevance of this enzyme as a target for AD treatment was also not clear.

Based on the foregoing, there is a strong need in the art for characterizing the function of eEF2K. There is a further need for determining its relevance with respect to AD and as a potential target site for AD treatments. The instant invention addresses these needs.

### SUMMARY OF THE INVENTION

The present invention relates to methods for preventing or treating Alzheimer's Disease in a patient by inhibiting the phosphorylative activity of eEF2K. It has now been discovered that eEF2K knock out mice crossed with transgenic mice expressing human genes linked to familial AD exhibit significantly less amyloid deposit development in their brains as they age. Specifically, the present invention provides methods for treating AD by inhibiting the deposit of amyloid plaques.

Therefore, according to one aspect of the present invention, a method is provided for inhibiting the build-up of amyloid plaques in the brain of a patient with risk factors for, or a diagnosis of Alzheimer's Disease, by administering to the patient an amount of one or more compounds effective to inhibit the phosphorylative activity of eEF2K, thereby inhibiting amyloid plaque deposition. In one embodiment, the eEF2K inhibitor is a competitive or noncompetitive inhibitor. In another embodiment, the eEF2K inhibitor is selected from:

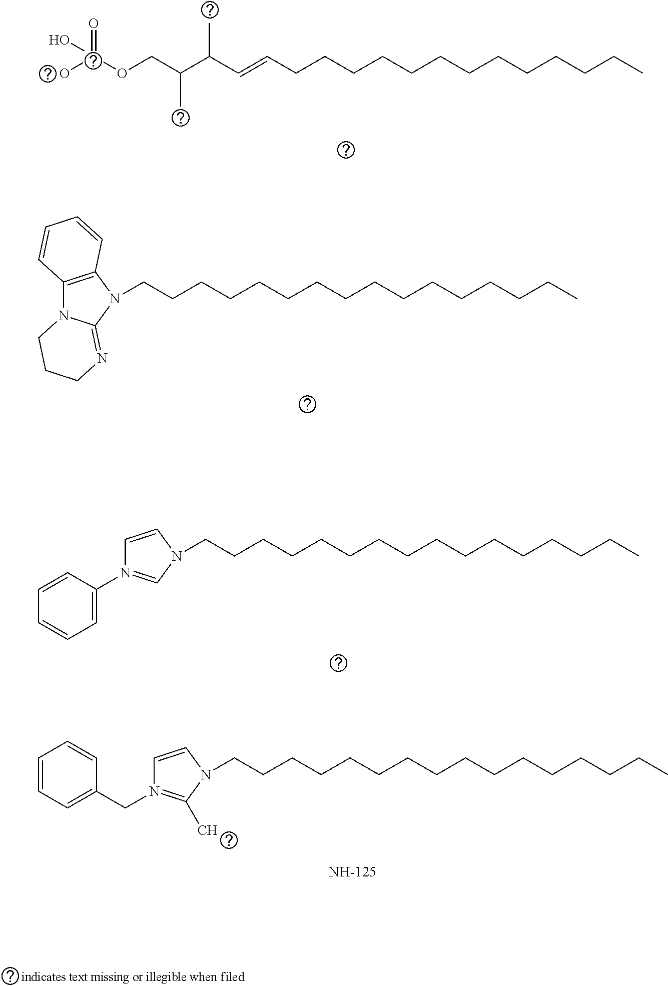

and combinations thereof.

In an alternative embodiment, the eEF2K inhibitor is selected from the group consisting of

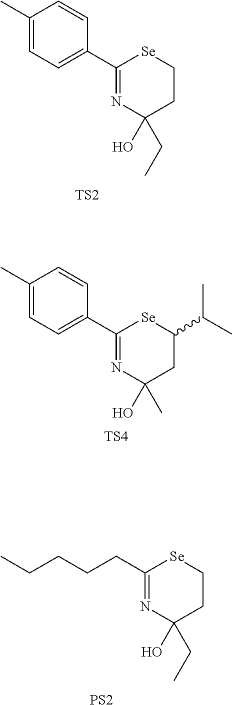

and combinations thereof.

In a further embodiment, the eEF2K inhibitor is a chalcone.

In a further embodiment, the eEF2K inhibitor is administered in a chronic dose. In an even further embodiment the eEF2K inhibitor is administered orally or intravenously.

### BRIEF DESCRIPTION OF THE DRAWINGS

FIG. 1 shows Hippocampal mGluR-LTD impaired in slices derived from Arc KO mice;

FIG. 2 shows Arc protein rapidly synthesized by group I mGluR activation and required for mGluR-dependent endocytosis of AMPA Receptors;

FIG. 3 shows eEF2K binds Homer and mGluR1/5;

FIG. 4 shows dynamic interaction of eEF2K and mGluR5;

FIG. 5 shows rapid induction of Arc by group I mGluRs dependent on eEF2K;

FIG. 6 shows mGluR-LTD impaired in hippocampal slices derived from eEF2K KO mice;

FIG. 7 shows LTD impaired in hippocampal slices derived from Arc/Fmr1 double KO mice;

FIG. 8 shows eEF2K, FMRP and rapid, de novo translation of Arc protein in mGluR-LTD;

FIG. 9 shows Western blots of detergent lysates from forebrains of APPswe/PS1ΔE9 transgenic mice that are either in WT background or in eEF2K KO background;

FIG. 10 shows the results of an ELISA determination of Aβ levels in 13-month-old APP/EF2K-KO mice compared with 12-month old APP/WT mice;

FIG. 11 shows plaque formation in 13-month-old APP/EF2K KO mice reduced compared to 12-month old APP/WT mice;

FIG. 12 shows reduction of plaque area in hippocampus of 13-month old APP/eEF2K KO vs. 12-month-old APP/WT mice;

FIG. 13 shows mGluR-LTD induced by high dose of DHPG impaired in Arc KO;

FIG. 14 shows rapid synthesis of Arc protein by activation of group I mGluRs;

FIG. 15 shows Arc mRNA detected in hippocampal dendritic regions of mice in an unstimulated state;

FIG. 16 shows Analysis of eEF2K interaction with Homer and mGluR5;

FIG. 17 shows eEF2K activity regulated by group I mGluRs;

FIG. 18 shows rapid induction of Arc protein by DHPG absent in eEF2K KO hippocampal neurons;

FIG. 19 shows characterization of Schaffer collateral-CA1 synapses of eEF2K KO. fEPSPs measured in the Schaffer collateral-CA1 synapses of eEF2K KO mice and compared to WT littermate controls;

FIG. 20 shows reduction of surface AMPAR by mGluR stimulation absent in eEF2K KO cultured neurons; and

FIG. 21 shows Characterization of Arc protein and Schaffer collateral-CA1 synapses of Fmr1 KO.

### DETAILED DESCRIPTION OF PREFERRED EMBODIMENTS

The present invention relates to methods for preventing or treating Alzheimer's Disease in a patient by inhibiting the activity of eEF2K. Specifically, the present invention provides methods for treating AD by inhibiting the build-up of insoluble Aβ and plaque load in a patient's brain through administration of a therapeutically effective amount of an eEF2K inhibitor.

As defined above, eEF2K belongs to a novel family of protein kinases, with the prototypical member being Dictyostelium myosin heavy chain kinase A, which displays little to no homology to conventional eukaryotic protein kinases. It is specific to eEF2 and is responsible for eEF2 phosphorylation, which promotes ribosomal translocation. As evident from the peptide screening assay discussed below, one consensus sequence for eEF2K phosphorylation is the amino acid sequence RKKYKFNEDTERRRFL (SEQ ID NO: 7). Phosphorylation of eEF2 was found to arrest translation, suggesting that this may be a critical mechanism by which the rate of protein synthesis is regulated. eEF2K was also previously shown to have increased activity in human brains of individuals with AD but, until the instant invention, the relevance of this as a mechanism for AD treatment was not clear.

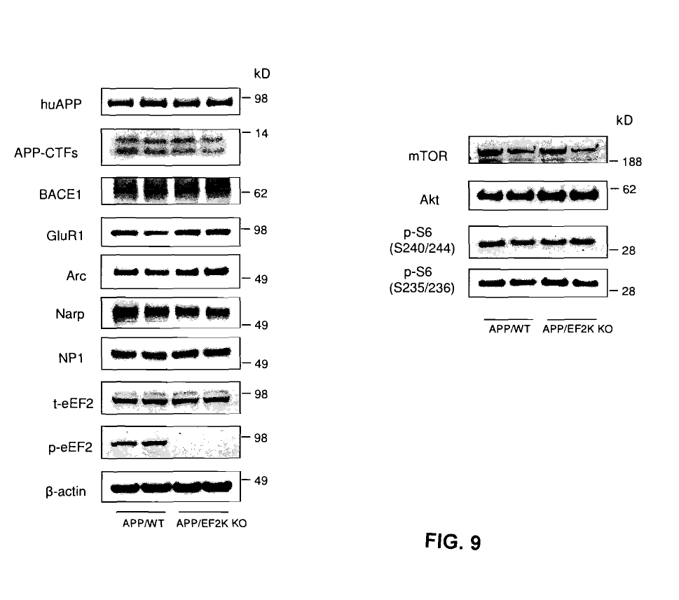

As shown in FIGS. 9-12 of the instant invention, and described in the Examples below, substantial reductions of insoluble forms of both Aβ40 and Aβ42 were observed in the brains of eEF2K-KO mice genetically altered to express these proteins. In other words, the net effect of inactivating eEF2K activity in organisms subject to amyloid plaque development is to inhibit the depositing of insoluble Aβ and the development of the associated plaque formations. To this end, administration of one or more compounds that modulate or inhibit eEF2K phosphorylative activity, similarly yield substantial reductions in the depositing of insoluble Aβ and the development of amyloid plaque formations.

In one embodiment, the eEF2K inhibitor of the present invention is a compound that either binds to or alters the kinase domain of eEF2K to prevent the enzyme from phosphorylating eEF2. To this end the inhibitor may competitively inhibit the phosphorylative activity of the eEF2K enzyme. Alternatively, the inhibitor may interact with the protein at a site other than the kinase domain, which alters the structure of the enzyme or otherwise causes kinase domain inactivation. To this end, the inhibitor may noncompetitively inhibit eEF2K phosphorylative activity.

In further embodiments, the eEF2K inhibitor is comprised of sphingosine-1-phosphate having the following structure:

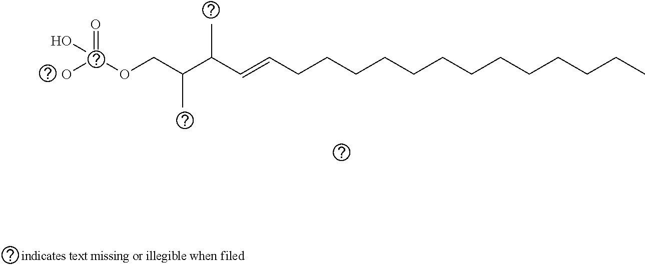

The eEF2K inhibitor also may be structurally similar to the sphingosine-1-phosphate, particularly with respect to the sixteen carbon aliphatic tail moiety and/or the positively charged head moiety. Non-limiting examples of such compounds may include L-587, L-207, or NH-125, which are comprised of the following respective structures:

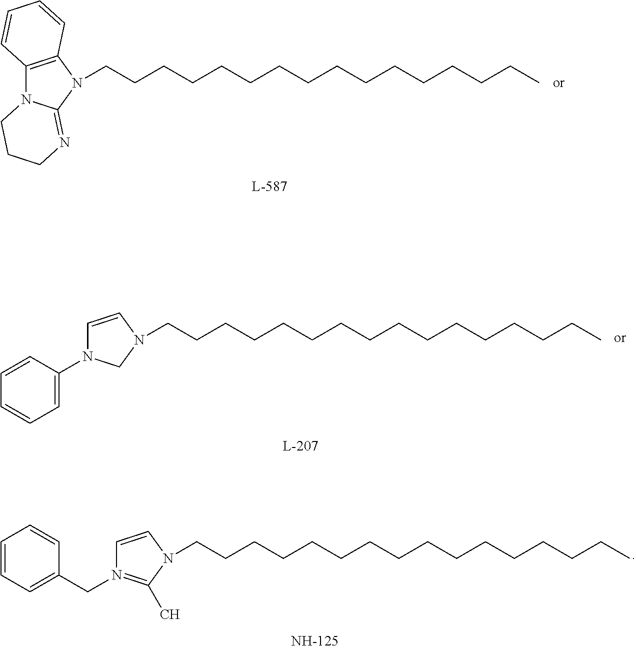

In further embodiments, the instant invention may include structural analogs of any of sphingosine-1-phosphate, L-587, L-207, or NH-125. As used herein, “analog” or “structural analog” refers to compounds having one or more atoms, functional groups, or substructures replaced or substituted with different atoms, groups, or substructures. Structural analogs of sphingosine-1-phosphate, L-587, L-207, or NH-125 may be comprised of a head region and a tail portion, and may be collectively represented by formula I:

Het-X-alk (I)

wherein Het is an optionally substituted aromatic or non-aromatic heterocyclic ring or ring system or an optionally N-substituted guanidine, X is either a direct bond or NH, and alk is an optionally substituted, saturated or unsaturated, straight chain or branched C14-C18 aliphatic tail. One or more carbons of the aliphatic tail may be substituted with one or more isosteric groups such as one or more aryl or heteroaryl moieties alone or as part of a ring system. Therapeutically valuable analogs having the structure of formula I, including compounds containing the optional substituents disclosed herein or other known pharmaceutical compound building blocks, may be identified using the screening methods discussed herein or with others known in the art.

Exemplified analog compounds consistent with formula I may include, but are not limited to, one or more of the following:

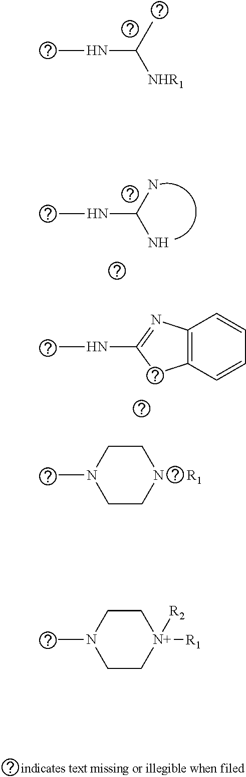

wherein the R substituents are independently selected from H, a straight or branched chain optionally substituted alkyl group, an optionally substituted cycloalkyl

Exemplified analogs consistent with formula I also include one or more of the following structures:

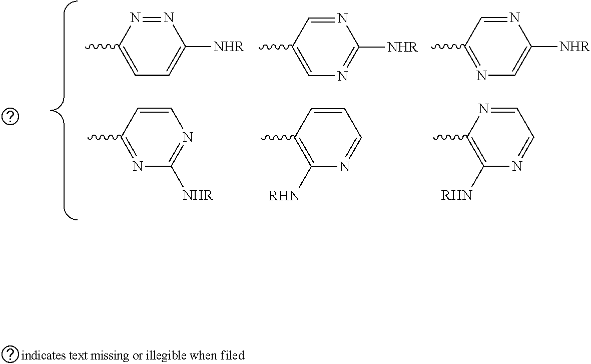

wherein the R substituents are also the same as described above for formula I.

In further embodiments, the eEF2K inhibitor may be comprised of a selenazine compound or an analog thereof. For example, in certain non-limiting embodiments, the eEF2K inhibitor is comprised of any one of the selenazine compounds TS2, TS4, or PS2, which are comprised of the following respective structures:

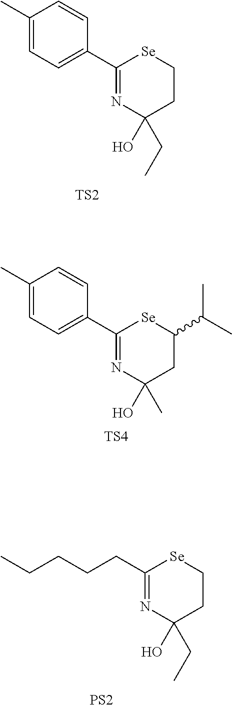

The selenazine compounds may also include analogs of the foregoing having a 1,3 selenazine core with one or more substituent groups extending therefrom. Such analogs may be collectively represented by formula II:

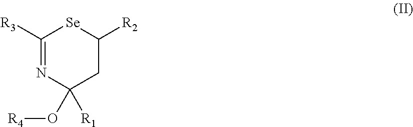

wherein R₁, R₂, R₃ and R₄ may be independently selected from H, a straight or branched chain optionally substituted alkyl group, an optionally substituted cycloalkyl group, and an optionally substituted aryl or heteroaryl group. The optional substituents may be selected from lower alkyl, lower alkoxy, nitro, —COOH, —NH-lower alkyl, —CO—NH-lower alkyl, —NH-acyl, and the like. R₄ may also include acyl and carboamyl groups. One of ordinary skill in the art will appreciate that therapeutically valuable analogs having the structure of formula II that are unsubstituted or contain the identified substituents or other pharmaceutical compound building block substituents may be identified using the screening methods discussed herein or with others known in the art.

In even further embodiments, the eEF2K inhibitor is comprised of chalcone, or analogs thereof. Rottlerin (IC50 4 ìM, Cho et al., 2000). In one embodiment, chalcone may be represented by the structure:

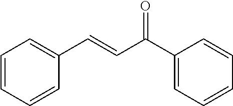

eEF2K inhibitory compounds of the present invention are identified using a high-throughput screening assays, such as the assay discussed herein and disclosed within U.S. Provisional Application No. 61/225,875, filed Jul. 15, 2009, the contents of which are incorporated herein by reference. Specifically, eEF2K can be produced in large quantities by E. coli, or using any other suitable means known in the art. Phosphorylation of a consensus sequence for eEF2K activity, such as Ac-RKKYKFNEDTERRRFL (SEQ ID NO: 7), can then be measured and compared with reduced activity seen in the presence of a test inhibitor compound. In one non-limiting embodiment, kinase activity is measured in both control and test batches based on the depletion of ATP. More specifically, active eEF2K utilizes ATP when phosphorlyating the consensus sequence. Thus, a reduction in ATP signals an active kinase. This may be visually detected and quantified by known methods, for example, by coupling the reaction with a luciferase luminescence assay, which is ATP dependent. Thus, active kinase will reduce ATP and, thereby, reduce the luminescence detected. Conversely, inhibition of eEF2K by a test compound prevents depletion of ATP, which is detected as an increased luminescence.

Any one or more of the foregoing compounds or analog compounds may be administered in therapeutically effective amount to a patient with risk factors for or a diagnosis of AD. Risk factors include the above-described age, family history, genetics, and head trauma. The term “effective amount” or “therapeutically effective amount” means that amount of a compound or agent that will elicit the biological or medical response of a subject that is being sought by a medical doctor or other clinician. In this case, the therapeutically effective amount would be the amount of the compound(s) or analog compound(s) effective to inhibit the phosphorylative activity of eEF2K, thereby inhibiting the deposit of Aβ and the development of amyloid plaques in the brain.

In the patient, the effect of the eEF2K inhibitor may be measured by evaluating alterations in the eEF2K pathway. In a non-limiting embodiment, this may be conducted, by evaluating the level of eEF2 phosphorylation in lymphocytes taken from a blood sample. For example, a phosphospecific antibody that recognizes only phosphorylated eEF2 may be used for such purposes. The effects of the eEF2K inhibitor in Alzheimer's patients may be further measured by tracking the patient's cognitive function and whether improvement results post-administration. Similar methods understood in the art may also be employed.

The eEF2K inhibitor may be administered in a single composition or dosage form or one or more compounds may be independently administered in separate compositions. Separate compositions may be administered simultaneously or sequentially. According to the methods of the present invention, the composition is administered systemically to a patient in need thereof. Systemic delivery may be accomplished through, for example, oral or parenteral administration. More specific routes of administration include intravenous, intramuscular, subcutaneous, intrasynovial, intraperitoneal, transmucosal, and transepithelial including transdermal and sublingual.

For parenteral administration, emulsions, suspensions or solutions of one or more eEF2K inhibitors in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are used. The injectable forms must be fluid to the extent that it can be easily syringed, and proper fluidity can be maintained, for example, by the use of a coating such as lecithin, by the maintenance of the required particle size in the case of dispersion and by the use of surfactants. Prolonged absorption of the injectable compositions can be brought about by use of agents delaying absorption, for example, aluminum monostearate and gelatin. The solutions of the salts of the products according to the invention are especially useful for administration by intramuscular or subcutaneous injection. Solutions of the eEF2K inhibitor as a free base or pharmacologically acceptable salt can be prepared in water suitably mixed with a surfactant such as hydroxypropyl-cellulose. Dispersions can also be prepared in glycerol, liquid polyethylene glycols, and mixtures thereof and in oils. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation, microfiltration, and/or by various antibacterial and antifungal agents, for example, parabens, chlorobutanol, phenol, sorbic acid, thimerosal, and the like.

Sterile injectable solutions are prepared by incorporating one or more active agents in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique, which yield a powder of the active ingredient plus any additional desired ingredient from previously sterile-filtered solution thereof.

One or more active agents may be also incorporated in a gel or matrix base for application in a patch, which would allow a controlled release of compound through transdermal barrier.

The percentage of one or more active agents in the compositions used in the present invention may be varied, it being necessary that it should constitute a proportion such that a suitable dosage shall be obtained. Several unit dosage forms may be administered at about the same time. A dose employed may be determined by a physician or qualified medical professional, and depends upon the desired therapeutic effect, the route of administration and the duration of the treatment, and the condition of the patient.

The terms “chronic dose” or “continuous administration” of the active agent(s) mean the scheduled administration of the active agent(s) to the patient on an on-going day-to-day basis.

In the adult, the doses are generally from about 0.01 to about 100, preferably 0.1 to 70, more especially 0.5 to 10, mg/kg body weight per day by oral administration, and from about 0.001 to about 10, preferably 0.01 to 10, mg/kg body weight per day by intravenous administration. In each particular case, the doses are determined in accordance with the factors distinctive to the patient to be treated, such as age, weight, general state of health and other characteristics, which can influence the efficacy of the compound according to the invention. The maximum dosage amount tolerated by the patient is preferred.

The active agent(s) used in the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active agent(s) may be administered 1 to 4 times per day. Of course, for other patients, it will be necessary to prescribe not more than one or two doses per day.

The eEF2K inhibitor can be administered during any stage (e.g. early, middle, or advanced) of AD or as a preventative for AD. Additionally, the eEF2K inhibitor can be administered in a chronic dose, for example, following an initial course of therapy.

The eEF2K inhibitor(s) of the present invention may also be administered in combination with other AD therapeutic agents otherwise known in the art. Such agents may include, but are not limited to, cholinesterase inhibitors such as donepezil, rivastigmine, galantamine, and tacrine; or glutamate inhibitors such as memantine and riluzole. To this end, the present invention also relates to the combination of an eEF2K inhibitor and any other agent capable of preventing or treating Alzheimer's disease.

The following non-limiting examples set forth hereinbelow illustrate certain aspects of the invention.

### EXAMPLES

### Materials and Methods

### AMPA Receptor Trafficking Experiments

Labeling of surface or internalized pool of AMPA receptor was performed as described with minor modifications (Shepherd, et al., Neuron, 52, 475-484 (2006)). Briefly, surface GluR1-containing AMPA receptors were then labeled by adding 2.5 μg of GluR1-N JH1816 pAb to the neuronal growth media and were subsequently incubated at 37° C. for 15 or 60 minutes after 5 min DHPG application. To visualize surface and internalized GluR1, Alexa 555 secondary was added in excess live at 10° C. Neurons were fixed, permeabilized and subsequently exposed to Alexa 488 secondary to stain internalized receptors (background in the non-permeabilized control was negligible).

### Electrophysiology

Field recording of excitatory postsynaptic potential (fEPSP) of hippocampal CA1 neurons of postnatal day (P)21-30 male mice was performed as described with minor modifications (Huber, et al., Science, 288, 1254-1257 (2000)). mGluR-LTD was induced by a mGluR1/5 agonist, (R,S)-3,5-DHPG for 5 min (Tocris, 50 μM, unless otherwise indicated), or by paired-pulse low-frequency stimulation (PP-LFS: 50-msec interstimulus interval, 1 Hz, for 15 min) in the presence of D-APV (Tocris, 50 μM). NMDAR dependent-LTD was induced by using 900 single pulses delivered at 1 Hz (Huber et al., 2000).

LTP was measured in Schaffer collateral-CA1 synapses in hippocampal slices derived from 8-10 week old male mice. Late phase-LTP (L-LTP) was induced by 4 trains of high frequency stimulation (HFS) (100 Hz, 1 sec) with 3 sec of intertrain interval.

### Antibodies

The following antibodies were previously described or obtained commercially: anti-phospho-eEF2 (Thr56: rabbit polyclonal) and total-eEF2 (rabbit polyclonal) from Cell Signaling; eEF2K (rabbit polyclonal) and mGluR1 (mouse monoclonal) from BD Biosciences; mGluR2 and PSD-95 from Upstate; mGluR4 from Zymed: horse radish peroxidase (HRP) conjugated HA antibody, HRP-conjugated myc antibody, myc (mouse monoclonal), and actin (mouse monoclonal) from Santa Cruz (Lyford, et al., Neuron, 14, 433-445 (1995)). mGluR5 and N-GluR1 antibodies were a kind gift from Richard L. Huganir.

### Constructs

The full-length mGluR1, mGluR5, and Homer cDNA constructs have been described previously (Tu et al., Neuron, 21, 717-726 (1998)). Full-length mGluR2 and mGluR4 was gifts from Dr. Paul Kammermeier (Northeastern Ohio University). HA and myc-tagged eEF2K constructs were prepared by polymerase chain reaction (PCR) using Pfu Turbo Polymer-ase (Stratagene) with specific primers containing SalI and NotI sites using the GST-eEF2K construct as a template. After digestion with SalI/NotI, PCR products were subcloned into an N-myc or N-HA-tagged pRK5 vector (modified from Genentech).

### Cell Culture

Neuronal cultures from embryonic day 18 (E18) pups were prepared as reported previously (Rumbaugh et al., J. Neurosci., 23, 4567-4576 (2003)), with minor alterations. For biochemistry experiments, 0.4×10⁶ neurons were added to each well of a 6-well plate (Coming) coated with poly-L-lysine. Growth medium consisted of NeuroBasal (Invitrogen) supplemented with 1% fetal bovine serum (Hyclone), 2% B27, 1% Glutamax (Invitrogen), 100 U/mL penicillin, and 100 U/mL streptomycin (Invitrogen). Neurons were fed twice per week with glia conditioned growth medium.

HEK293T cells were maintained in Dulbecco's modified Eagle's medium (DMEM) with GlutaMAX, containing 10% heat-inactivated fetal bovine serum (Invitrogen), 100 U/mL penicillin, and 100 U/mL streptomycin at 37° C. and 5% CO₂.

### Immunoprecipitation (IP) Assays

Synpatoneurosomes from mouse forebrains were prepared as described (Scheetz, et al., Nat. Neurosci., 3, 211-216 (2000) and Takei et al., J. Neurosci., 24, 9760-9769 (2004)), with modifications. Mouse brain tissues were dissected and homogenized four times with a Dounce homogenizer in 6 ml homogenization buffer (50 mM HEPES, pH 7.4, with 119 mM NaCl, 4.7 mM KCl, 1.18 mM MgSO₄, 1.18 mM KH₂PO₄, 24.9 mM NaHCO₃, 10 mM glucose, and 1.3 mM CaCl₂,) containing Complete™ EDTA-Free protease inhibitors (Roche). The homogenate was passed through two layers of 100 μm and 50 μm nylon mesh filter (Millipore) and one layer of 10 μm Mitex filter (Millipore). Heavy particles were removed by brief centrifugation (1,000 g for 30 sec at 4° C.). The supernatant was collected and centrifuged (1,000 g for 10 min at 4° C.), and the pellet was resuspended with five volumes of DMEM.

Synaptosome preparations (100 μl ) were treated with 100 μM (final concentration) of DHPG (Tocris) or mock-treated with water and incubated at 37° C. for 20 min. Five hundred microliters of IP buffer (1× PBS, pH 7.4, with 5 mM EDTA, 5 mM EGTA, 1 mM Na₃VO₄, 10 mM sodium pyrophosphate, 50 mM NaF, and 1% Triton X-100) containing Complete™ EDTA-Free protease inhibitors was added and vigorously vortexed.

The supernatant (300 μl) was then mixed with 0.5-2 μg of the appropriate antibody for 3 hours at 4° C. Then 50 μl of 1:1 protein A- or protein G-Sepharose slurry (Amersham-Pharmacia Biotech) was added for an additional 1 h. The protein beads were washed three times with IP buffer containing 1% Triton X-100. The protein samples were eluted with 80 μl of SDS loading buffer and analyzed by gel electrophoresis and Western blotting.

HEK293T cells grown in 6-well plates to 30% confluence were transfected with 0.5 μg cDNA each per well, using the FuGENE 6 transfection reagent according to the manufacturer's protocol (Roche). After 2 days, cells were harvested in 0.4 ml IP buffer containing 1% Triton X-100 and Complete™ EDTA-Free protease inhibitors. The lysate was sonicated six times for 0.4 sec each, and then centrifuged at 13,200 rpm for 15 min at 4° C. in a tabletop centrifuge. Supernatants (300 μl) were used for IP assays as described above.

A computer program was used to titrate the concentration of Ca²⁺ in IP buffer. (http://www.stanford.edukpatton/muc.html).

### Western Blotting

Transfected HEK293T cells or cultured neurons were treated with various drugs and then harvested in IP buffer supplemented with 1% Triton X-100 buffer and Complete™ EDTA-Free protease inhibitors. Soluble fractions were diluted with 4× SDS sample buffer.

Samples were separated electrophoretically using NuPAGE 4-12% Bis-Tris gels (Invitrogen) and transferred to an Immobilon-P PVDF membrane (Millipore). The membrane was blocked with TBST (50 mM Tris, pH 7.5, with 150 mM NaCl, and 0.1% Tween-20) containing 5% non-fat milk for one hour at room temperature, followed by incubation with primary antibody in TBST buffer overnight at 4° C. After three washes with TBST buffer, membranes were incubated with HRP-conjugated anti-rabbit, or anti-mouse antibody in TBST for another hour. After three washes with TBST buffer, the membrane was treated with SuperSignal ECL substrate (Pierce) according to the manufacturer's protocol.

To reduce the background signal in the co-IP assay, HRP-conjugated HA or HPR conjugated myc antibody (Santa Cruz) was used when overexpressed proteins were tagged with HA or myc. When cultured neuronal samples were used for Western blot analysis with rabbit polyclonal antibodies such as α-Arc, α-phospho-eEF2, or α-eEF2, HRP-conjugated protein A (Amersham-Pharmacia Biotech) was used instead of HRP-conjugated rabbit secondary antibody; this approach helped minimize the non-specific signal around 70 kDa. Image J software (NIH) was used for quantification.

### Immunocytochemistry and Immunohistochemistry

Immunocytochemistry of cultured neurons was performed as described (Shepherd et al., Neuron, 52, 475-484 (2006)). Briefly, DIV14 primary hippocampal neurons were fixed in fixation solution (4% paraformaldehyde, 4% sucrose containing 1× PBS) for 20 min at 4° C. and were permeabilized with 0.2% Triton X-100 in PBS for 10 min 4° C. Cells were then blocked for 1 hr at room temperature in 10% normal goat serum (NGS). Primary antibodies were diluted (1:250 of phospho-eEF2 antibody, 1:500 of PSD95, 1:300 for Arc) in 10% NGS and incubated with neurons for overnight at 4° C. Alexa 488, or Alexa 555-conjugated secondary antibodies (1:500; Molecular Probes) were diluted in 10% NGS and incubated at room temperature for 1 hr. Coverslips were mounted with PermaFluor containing DAPI (Invitrogen). All images were taken with same exposure and setting using Zeiss 510 Meta confocal microscope. Quantification of Arc levels was carried out using Image J software. For the measurement of dendritic Arc levels, average pixel intensity was measured in the primary dendrites 20 μm away from the soma.

Immunohistochemistry of phospho-eEF2 in WT and eEF2K mice was performed as described (Ramirez-Amaya et al., J. Neurosci. 25, 1761-1768 (2005)) with slight modifications. Hippocampal slices were prepared and stimulated with DHPG as described in an Electrophysiology section. After stimulation, slices were immediately frozen with ethanol-dry ice solution. Twenty micrometer sections were prepared in optimal cutting temperature compound (Sakura, Tokyo, Japan) and were mounted on superfrost-coated slides. The sections were fixed in ice-cold fixation solution (2% paraformalde-hyde, 7.4 pH) for 10 mm and washed in 2×SSC, pH 7.0. Incubation of slides with 50:50% acetone/methanol for 8 min at 4° C. was followed by washing in 2×SSC containing 0.05% Tween 20 and quenching in 2×SSC and 1% H₂O₂ for 20 min. Slices were blocked with tyramide signal amplification kit (TSA) blocking buffer (PerkinElmer Life Sciences), and were incubated in phospho-eEF2 antibody (1:250) for 48 hr at 4° C. Incubation with the anti-rabbit biotinylated secondary antibody (Vector Laboratories) for 2 hr at room temperature was followed by amplification with the avidin-biotin system (Vector Laboratories) for 1 hr. The signals were visualized using the cyanine 3 (CY3) TSA fluorescence system (PerkinElmer Life Sciences), and the nuclei were stained with DAPI (Molecular Probes). No staining was detected in the absence of the primary or secondary antibodies. No phospho-eEF2 signal was detected in eEF2K KO sections.

### AMPA Receptor Trafficking Experiments

DIV 14-21 mouse primary hippocampal cultures were incubated in neuronal growth media containing 50 μMDHPG for 5 minutes and then washed with new growth media. Surface GluR1-containing AMPA receptors were then labeled by adding 2.5 μg of GluR1-N JH1816 pAb to the neuronal growth media and were subsequently incubated at 37° C. for 15 or 60 minutes after 5 min DHPG application. To visualize surface and internalized GluR1, Alexa 555 secondary was added in excess live at 10° C. Neurons were fixed, permeabilized and subsequently exposed to Alexa 488 secondary to stain internalized receptors (background in the non-permeabilized control was negligible).

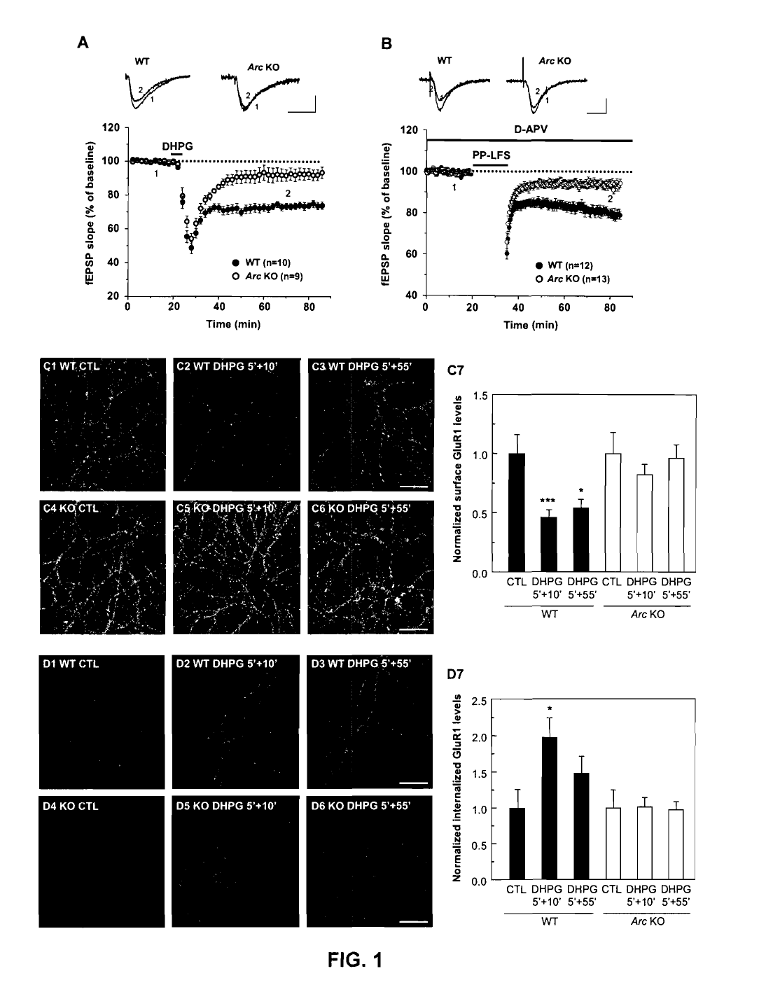

Quantification of surface GluR1 puncta was carried out using Image J software. Images were acquired as multi-channel TIFF files with a dynamic range of 4096 gray levels (12-bit binary; MultiTrack acquisition for confocal) using metamorph software on a Zeiss LSM 510 confocal microscope. To measure punctate structures neurons were thresholded by gray value at a level close to 50% of the dynamic range. Background noise from these images was negligible. All puncta were treated as individual objects and the characteristics of each, such as area and average fluorescence, were logged measured. The Data reflected in FIGS. 1B-C was an example of one representative experiment. The total intensity of GluR1 levels were normalized to control, untreated neurons at 60 min. Significance was determined by a paired Student's t-test.

### Electrophysiology

Field recording of excitatory postsynaptic potential (fEPSP) of hippocampal CA1 neurons of postnatal day (P)21-30 male eEF2K KO mice (129XC57B1/6), Arc KO mice (C57B1/6), Fmr1 KO mice (FVB) and their WT littermates was performed as described with minor modifications (Huber et al., Science, 288, 1254-1257 (2000)). Hippocampal slices were prepared in ice-cold dissection buffer (212 mM sucrose, 2.6 mM KCl, 1.25 mM NaH₂PO₄, 26 mM NaHCO₃, 5 mM MgCl₂, 0.5 mM CaCl₂, and 10 mM dextrose). Slices were recovered for 2.5 h at 30° C. in artificial cerebrospinal fluid (ACSF: 124 mM NaCl, 5 mM KCl, 1.25 mM NaH₂PO₄, 26 mM NaHCO₃, 1 mM MgCl₂, 2 mM CaCl₂, and 10 mM D-glucose) saturated with 95% O₂, 5% CO₂. For recording, slices were placed in a submersion recording chamber and perfused with 30° C. ACSF at a rate of 2 ml/min.

fEPSPs were recorded with extracellular recording electrodes (1.0 MΩ) filled with ACSF and placed in the stratum radiatum of area CA1. Synaptic responses were evoked by a 200-μsec current pulse to Schaffer collateral axons with a concentric bipolar tungsten stimulating electrode. Stable baseline responses were collected every 30 sec by using a stimulation intensity (10-30 μA) yielding 50-60% of the maximal fEPSP slope response.

mGluR-LTD was induced by a mGluR1/5 agonist, (R,S)-3,5-DHPG (Tocris), or by electrical stimulations. DHPG (50 μM, unless otherwise indicated) was perfused at a rate of 2 ml/min for 5 min. mGluR-LTD was electrically induced in the presence of the N-methyl-D-aspartate (NMDA) receptor antagonist D-(−)-2-amino-5-phosphono-valenic acid (D-APV) (Tocris) (50 μM) by using paired-pulse low-frequency stimulation (PP-LFS), consisting of 900 pairs of stimuli (50-msec interstimulus interval) delivered at 1 Hz. NMDAR dependent-LTD was induced by using 900 single pulses delivered at 1 Hz.

LTP was measured in Schaffer collateral-CA1 synapses in hippocampal slices derived from 8-10 week old male mice as described (Young et al., Eur. J Neurosci., 23, 1784-1794 (2006)). Late phase-LTP (L-LTP) was induced by 4 trains of high frequency stimulation (HFS) (100 Hz, 1 sec) with 3 sec of intertrain interval. fEPSPs were monitored for 3 hours following the induction of L-LTP.

### Real-Time Reverse Transcription-Polymerase Chain Reaction (RT-PCR)

RNA Extraction and cDNA Synthesis

RNA samples from neuronal cultures were prepared using the PARIS kit (Ambion) according to the manufacturer's protocol. Following RNA extraction, samples were treated with DNase to remove contaminating DNA prior to cDNA synthesis. Total RNA was reverse transcribed using the SuperScript II First Strand Synthesis System for RT-PCR (Invitrogen) according to the manufacturer's protocol. A negative control without reverse transcriptase was included.

### Primers and Real-Time PCR

The primer sequence for GAPDH:

(SEQ ID NO: 1)
5′-CTGGAGAAACCTGCCAAGTA-3′ (forward),
(SEQ ID NO: 2)
5′-AGTGGGAGTTGCTGTTGAAG-3′ (reverse).

The primer sequence for Arc:

(SEQ ID NO: 3)
5′-TGAGACCAGTTCCACTGATG-3′ (forward),
(SEQ ID NO: 4)
5′-CTCCAGGGTCTCCCTAGTCC-3′ (reverse)

Primer specificity was verified by melt curve analysis. PCR amplification of cDNA was performed using the BioRad iCycler Real-Time Detection System (BioRad Laboratories). cDNA (1 μl) was added to 24 μl of 1× reaction master mix (3 mM MgCl2, KCl, Tris-HCl, iTaq DNA polymerase, 25 units/ml SYBR Green 1, 0.2 mM each dNTPs, 10 nM fluorescein and 500 nM each gene specific primers). For each experimental sample, duplicate reactions were conducted in 96-well plates (BioRad). PCR cycling conditions consisted of a hot-start activation of iTaq DNA polymerase at 95° C. and 40 cycles of denaturation (95° C., 30 s), annealing (56° C., 30 s), and extension (72° C., 30 s). A melt curve analysis was conducted to determine the uniformity of product formation, primer-dimer formation, and amplification of non-specific products. PCR product was denatured (95° C., 1 min) prior to melt curve analysis, which consisted of incrementally increasing reaction temperature by 0.5° C. every 10 s from 60° C. to 95° C. All primers generated a single amplification product at a temperature above 80° C. (data not shown).

GAPDH was used to normalize data. The threshold for detection of PCR product above background was set at 10× the standard deviation of the mean background fluorescence for all reactions. Background fluorescence was determined from cycles 1-5 prior to the exponential amplification of product and subtracted from the raw fluorescence of each reaction/cycle. Threshold for detection of PCR product fell within the log-linear phase of amplification for each reaction. Threshold cycle (CT; number of cycles to reach threshold of detection) was determined for each reaction.

Relative gene expression was determined using the 2^{-ΔΔCT} method (Livak, et al., Methods 25, 402-408 (2001)). The mean CT of duplicate measures was computed for each sample and the sample mean CT of GAPDH (the internal control) was subtracted from the sample mean CT of Arc (ΔCT). The average CT of the samples from control neurons for Arc and GAPDH were then subtracted from the mean ΔCT of each experimental sample (ΔΔCT). 2^{-ΔΔCT} yields fold change in gene expression of the gene of interest normalized to the GADH gene expression and relative to the untreated control sample.

### Fluorescent In-Situ Hybridization and Confocal Microscopy

Mice were sacrificed immediately from their home cage by 30 sec exposure to isoflurane and decapitation. In-situ hybridization was performed as previously described (Guzowski et al., Nat. Neurosci., 2, 1120-1124 (1999)). Briefly, brains were rapidly removed and quick-frozen in a beaker of isopentane equilibrated in a dry ice/ethanol slurry and stored at −80° C. until further processing. Coronal brain sections (20 μm) were prepared using a cryostat and arranged on slides (Superfrost Plus, VWR) so that all experimental groups were represented on each slide. Slides were air dried and stored frozen at −20° C. until use. Slide-mounted brain sections were fixed in 4% buffered para-formaldehyde, treated with 0.5% acetic anhydride/1.5% triethanolamine, and equilibrated in 2×SSC. Slides were incubated in 1× prehybridization buffer (Sigma) for 30 min at room temperature. Arc riboprobe labeled with Fluorescein-UTP (100 ng) was diluted to 100 μl in a commercial hybridization buffer (Amersham), heat denatured, chilled on ice, and then added to each slide and hybridization was carried out at 56° C. for 16 hrs. Slides were washed to a final stringency of 0.5×SSC at 56° C. Endogenous peroxidase activity was quenched with 2% H₂0₂ in PBS, slides were incubated with the appropriate horseradish peroxidase (HRP)-antibody conjugate (Roche Molecular Biochemicals) 2 hrs at room temperature. Slides were washed three times in Tris-buffered saline (with 0.05% Tween-20), and the conjugate was detected using FITC-TSA fluorescence system (Perkin Elmer Life Sciences) and counterstained with DAPI. Slides were coverslipped with anti-fade media (Vectashield; Vector Labs, Burlington, Calif.) and sealed.

Stained slides were analyzed using a Zeiss LSM 510 confocal microscope. PMT assignments, pinhole sizes and contrast values were kept constant across different confocal sessions. Areas of analysis were z sectioned in 1.0-micron optical sections. Z-section image series were collected.

### Metabolic Labeling

To measure the incorporation of ³⁵S methionine and cysteine into new peptide, 30 μl protein labeling mix (Perkin Elmer) was added into 1.5 ml of regular culture medium (final 220 μCi/ml) for the time indicated in the figures. After washout once with ice-cold PBS, cells were lysed with 700 μl of RIPA buffer. After quantification of total amount of protein, equal amount of lysate (˜200 μl) was precipitated with 10% TCA.

### Surface Biotinylation Assay

For surface biotinylation, drug-treated cortical neurons were cooled on ice, washed twice with ice-cold PBS++ (1× PBS, 1 mM CaCl₂, 0.5 mM MgCl₂) and then incubated with PBS++ containing 1 mg/ml Sulfo-NHS-SSBiotin (Pierce) for 30 min at 4° C. Unreacted biotin was quenched by washing cells three times with PBS++ containing 100 mM Glycine (pH 7.4) (briefly once and for 5 min twice). Cultures were harvested in RIPA buffer and sonicated. Homogenates were centrifuged at 132,000 rpm for 20 min at 4° C. Fifteen % of supernatant was saved as the total protein. The remaining 85% of the homogenate was rotated with Streptavidin beads (Pierce) for 2 hr. Precipitates were washed with RIPA buffer three times (5 min each time). All procedures were done at 4° C.

### Example 1

### mGluR-LTD and PP-LFS LTD Require Arc

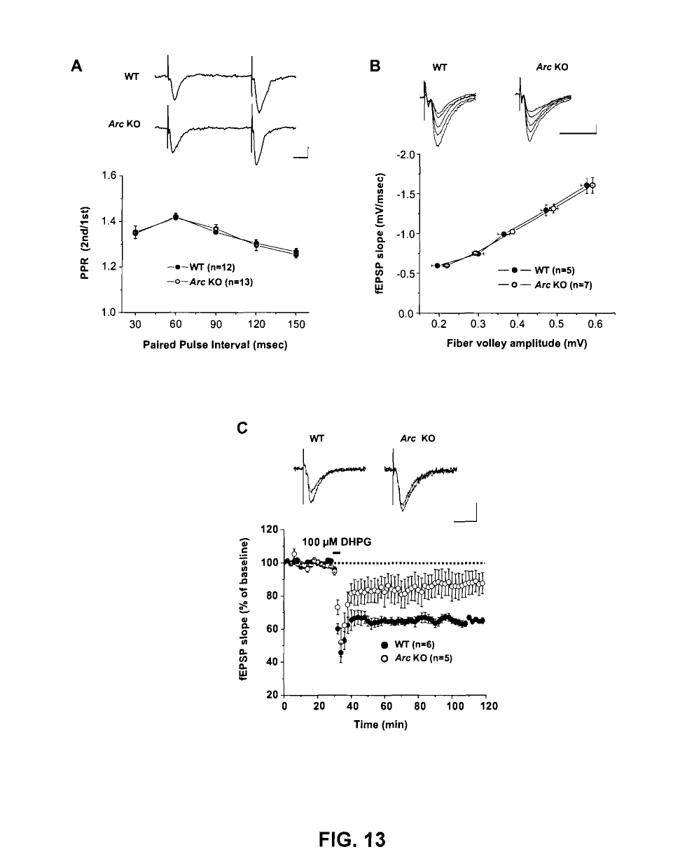

To examine the role of Arc in mGluR Long Term Depression (LTD) the Schaffer-CA1 synapses in acute hippocampal slices prepared from wild type (WT) and Arc knock-out (KO) mice were monitored. Baseline synaptic properties, including the fiber volley-fEPSP relationship (an index of basal synaptic strength) and paired pulse facilitation ratio were normal in Arc KO mice (FIGS. 13A and B), confirming a previous report (Plath et al., Neuron, 52, 437-444 (2006)). In WT slices treatment with the group I mGluR agonist, DHPG (50 μM) for 5 min followed by washout, produced a stable reduction of synaptic strength (72.8±2.0% of baseline, mean±standard error of the mean) (FIG. 1A). Synaptic stimulation using the paired-pulse low frequency stimulation (PP-LFS) protocol in the presence of the NMDA receptor antagonist, D-APV (50 μM) resulted in a similar stable reduction of synaptic strength to 79.9±2.1% of baseline (FIG. 1B).

In Arc KO slices, treatment with DHPG (92.1±3.7% of baseline, p<0.001 compared to littermate WT controls by unpaired two-tailed Student's t-test) or PP-LFS (94.3±2.1% of baseline, p<0.0001) failed to evoke robust LTD, albeit there is a slight residual LTD in Arc KO slices (p=0.09 for DHPG-LTD; p=0.03 for PP-LFS LTD by paired t-test). The residual LTD suggests that an Arc-independent pathway also contributes to mGluR-LTD. The immediate short-term synaptic depression during the induction period with DHPG and immediately following the PP-LFS protocol was not significantly different between WT and KO mice (FIGS. 1A and 1B). Furthermore, mGluR-LTD induced by higher concentration of DHPG (100 μM) was also impaired in Arc KO slices (88.5±7.4% of baseline for Arc KO slices; 64.4±2.6% of baseline for WT slices, p<0.01), indicating that the requirement for Arc does not depend on specific range of mGluR activation (FIG. 13C).

FIG. 1 shows Hippocampal mGluR-LTD impaired in slices derived from Arc KO mice; Field excitatory postsynaptic potentials (fEPSPs) were recorded in the hippocampal Schaffer collateral-CA1 synapses derived from Arc KO mice and compared to WT littermate controls. (A) Average time course of the change in fEPSP slope induced by the group I mGluR agonist, (R,S)-DHPG (50 μM, for 5 min). LTD of WT mice was 72.8±2.0% of baseline at t=70 min (n=10). In Arc KO, fEPSPs were 92.1±3.7% of the baseline at t=70 min (n=9). p<0.001 when compared to littermate WT. Error bars indicate the standard error of the mean. Measurements correspond to the time points indicated on the time-course graph in this and all subsequent figures. (B) Time course of the change in fEPSP slope produced by paired pulse low frequency stimulation (PP-LFS: at 1 Hz, 50 msec interstimulus interval, for 15 min) in the presence of the NMDA receptor antagonist, D-APV (50 μM). LTD of WT mice was 79.9±2.1% of baseline at t=80 min (n=12). In Arc KO mice, fEPSPs were 94.3±2.1% of the baseline at t=80 min (n=13). p<0.0001 Scale bars=0.5 mV/10 ms.

(C) 5 minutes of DHPG application resulted in a loss of surface GluR1 at 15 min (n=20, *** p<0.005) and 60 min (n=19, * p<0.05) after DHPG application, compared to untreated controls in WT hippocampal cultures. Arc KO neurons did not exhibit any changes in surface GluR1 levels after DHPG treatment. Representative pictures of cultures are shown using a LUT scale where white is high intensity and dark red is low intensity. (D) 5 minutes of DHPG application resulted in an increase of internalized GluR1 at 15 min (n=20, * p<0.05), compared with untreated cultures. Arc KO neurons did not exhibit changes in internalized GluR1 levels after DHPG treatment.

FIG. 13 shows mGluR-LTD induced by high dose of DHPG impaired in Arc KO; where (A) Relationship between paired-pulse interval and paired-pulse ratio (PPR) of the Schaffer collateral-CA1 synapses of WT and Arc KO mice. Exemplar traces are shown with 60 msec interval. (B) Relationship between fiber volley amplitude and fEPSP slope of the Schaffer collateral-CA1 synapses of WT and Arc KO mice. Each point represents the mean for a narrow range of fiber volley amplitudes. (C) Average time course of the change in fEPSP slope induced by DHPG (100 μM, for 5 min). LTD of WT mice was 64.4±2.6% of baseline at t=90 min (n=6). In Arc KO, fEPSPs were 88.5±7.4% of the baseline at t=90 min (n=5). p<0.01 when compared to littermate WT. Scale bars=0.5 mV/10 ms.

### Example 2

### mGluR-Dependent AMPA Receptor Endocytosis Requires Arc

mGluR1/5 activation results in a rapid and sustained loss of surface AMPARs that underlies synaptic depression. Since Arc KO mice have deficient mGluR-LTD, whether Arc is required for mGluR-dependent AMPAR endocytosis was directly tested. DHPG (50 μM) was applied to DIV 14-21 primary hippocampal neurons for 5 min followed by washout, and surface and internalized AMPARs were measured 15 mm or 60 min after DHPG application. In WT cultures DHPG resulted in a significant loss of surface GluR1 at 15 min and 60 min as compared with untreated cultures (FIGS. 1C1-3 and 1C7). However, GluR1 surface levels were unchanged after DHPG application in Arc KO neurons (FIGS. 1C4-6 and C7). WT cultures exhibited a significant increase in internalized GluR1 at 15 min (FIGS. 1D1-3 and D7). Arc KO neurons did not exhibit an increase in internalized receptors after DHPG application (FIGS. 1D4-6 and D7). Thus, Arc is required for rapid, mGluR-dependent AMPAR endocytosis.

### Example 3

### mGluR Induces Rapid Translation of Preexisting Arc mRNA

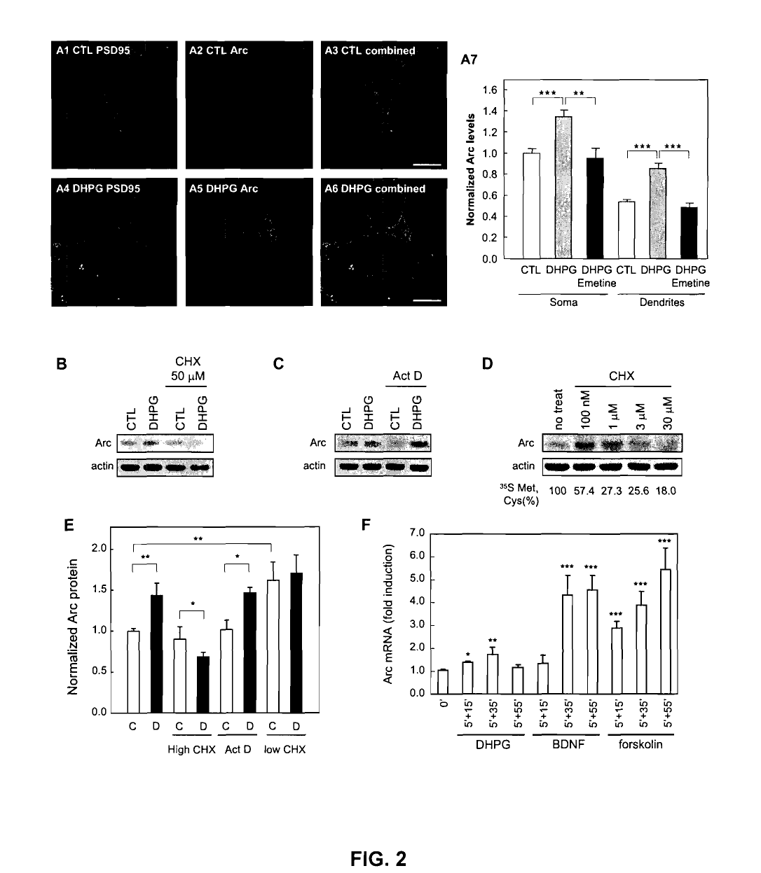

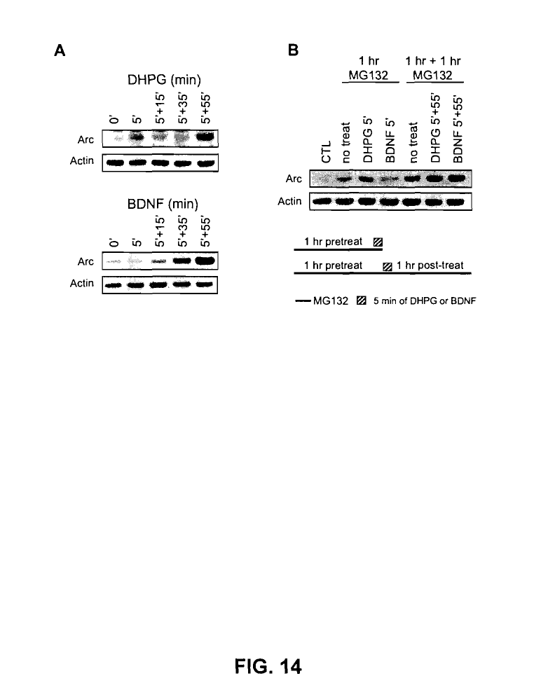

If Arc plays a direct role in mGluR-LTD, protein level should be acutely regulated in dendrites. Therefore, Arc protein expression was examined by immunocytochemistry in DIV14 hippocampal cultures. The basal level of Arc protein was low, but increased significantly in both the soma and dendrites during the 5 min incubation with DHPG (50 μM) (FIG. 2A). The increase of Arc protein was blocked by the protein synthesis inhibitor emetine, indicating a role for de novo translation. The induced Arc immunoreactivity in both proximal and distal dendrites was detected within 5 min of mGluR activation, and there was no evidence of a concentration gradient that might occur with rapid transport of Arc from the soma. The rapidity and distribution of the response suggests that Arc is synthesized locally in dendrites, and is consistent with the observation that mGluR-LTD is expressed in isolated dendrites. Similar levels of Arc induction during 5 min incubation of DHPG were observed by western blot analysis using forebrain cultures (FIG. 14A). Treatment with BDNF (10 ng/ml) also increased Arc protein expression, but in contrast to DHPG, this was evident only after 40 min (FIG. 14A).

The rapid increase of Arc protein could be mediated by an enhanced rate of translation, or a stable level of translation together with reduced degradation. As reported previously (Rao et al., Nat. Neurosci., 9, 887-895 (2006)) the proteosome inhibitor MG132 increased Arc protein, but did not block the ability of DHPG to further increase Arc (FIG. 14B). Induction of Arc by DHPG at 5 min was blocked by 5 min pretreatment of emetine or cycloheximide (FIGS. 2B and 2E). These data support the notion that Arc induction following DHPG treatment involves an increase in the rate of de novo protein translation.

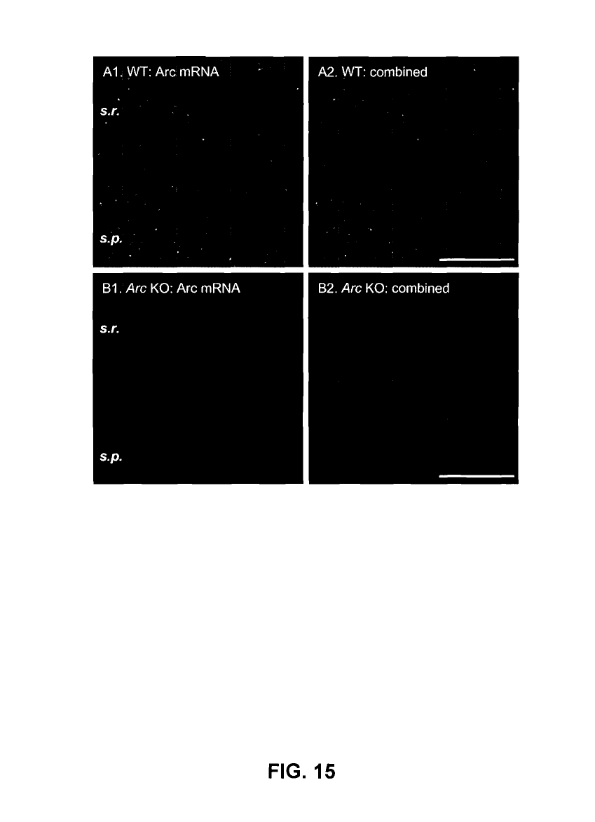

To examine the possible role of de novo transcription of Arc mRNA, the effect of the transcription blocker, actinomycin D was monitored. Actinomycin D (10 μM, 5 min pretreatment and 5 min with or without DHPG) did not alter the DHPG-induced increase of Arc protein (FIGS. 2C and 2E). DHPG did evoke a modest increase of Arc mRNA, but this was detected only after 20 min (FIG. 2F). The time course of the delayed Arc protein expression by DHPG or BDNF correlated with the mRNA induction, and actionmycin D blocked this response (data not shown). The observations suggest that the rapid increase in de novo translation requires Arc mRNA that is present in neurons prior to DHPG stimulation, while the delayed Arc expression is coupled to de novo transcription. Arc mRNA is detected in dendrites of unstimulated cultured neurons, Arc mRNA was detected in stratum radiatum of the hippocampal CA1 region from home-caged mice (FIG. 15).

FIG. 2 shows Arc protein rapidly synthesized by group I mGluR activation and required for mGluR-dependent endocytosis of AMPA Receptors; (A) Stimulation of hippocampal neurons with DHPG (50 μM) for 5 min increased Arc immunoreactivity in both cell body (1.34±0.063 of untreated soma, n=13) and dendrites (1.58±0.095 of untreated dendrites, n=38). The rapid increase of Arc was blocked by protein synthesis inhibitor, emetine (10 ng/ml, 10 min). (B) High dose cycloheximide (CHX, 50 μM, total 10 min: 5 min pretreatment and 5 min with or without DHPG) blocked the induction of Arc by DHPG (5 min). (C) Transcription inhibitor, Actinomycin D (ActD: 10 μM, 5 min pretreatment and 5 min with or without DHPG) did not block the induction of Arc by DHPG (5 min) (D) Low dose CHX increased the level of Arc protein. Neurons were treated with vehicle or various doses of CHX for 10 min.

Total protein synthesis was measured by counting the incorporation of ³⁵S methionine and cysteine in TCA precipitant. (E) Statistical analysis of Western blots. Five minute treatment of DHPG significantly increased the level of Arc. Inhibition of new protein synthesis by high dose of cycloheximide not only blocked the induction of Arc protein but also slightly decreased the level of Arc upon stimulation with DHPG. Inhibition of transcription by Actinomycin D did not affect the level of Arc. Low dose CHX (50-100 nM, 5 min pretreatment and 5 min with or without DHPG) increased the level of Arc, which was not further induced by DHPG. * p<0.05, ** p<0.01. (F) The level of Arc mRNA was measured using real-time RT-PCR. Stimulation of neurons with BDNF (10 ng/ml) and forskolin (50 μM) induced the level of Arc mRNA 40 min and 20 min after stimulation, respectively. DHPG slightly increased the level of Arc mRNA at 20 and 40 min after stimulation. * p<0.05, ** p<0.01, *** p<0.005.

FIG. 14 shows rapid synthesis of Arc protein by activation of group I mGluRs; DIV14 forebrain neurons were treated with either DHPG (50 μM) or BNDF (10 ng/ml) for 5 min, or subsequently incubated in the original medium without DHPG or BNDF until the time indicated. (A) Arc protein was induced by DHPG during the 5 min stimulation. It reached its highest point at 60 min after stimulation. BDNF increased the level of Arc protein 40 min after treatment. But, no change was seen after 5 min. (B) Proteasome inhibitor increased the basal level of Arc protein but did not occlude Arc induction by DHPG or BDNF. Neurons were pretreated with MG132 (10 μM), a proteasome and calpain inhibitor, for 1 hr and were stimulated with DHPG or BDNF. DHPG increased the level of Arc protein both in 5 min and 60 min after stimulation, while BDNF increased the Arc protein level only in 60 min.

FIG. 15 shows Arc mRNA detected in hippocampal dendritic regions of mice in unstimulated state; Arc mRNA was detected in the stratum pyramidal (s.p.), and stratum radiatum (s.r) of the hippocampal CA1 region from WT (A1 and A2) but not in Arc KO animals (B1 and B2) that were sacrificed immediately upon removal from their home cage. Blue and green colors show DAPI and Arc mRNA, respectively. Projected images composed of 20 Z-stacks taken at 1 μm interval are shown. Scale bar indicates 50 μm.

### Example 4

### Low Dose Cycloheximide can Increase Arc Protein Expression

In examining the dose-dependence of cycloheximide's actions, the level of Arc protein rapidly increased when neurons are treated with low doses (FIG. 2D). For example 100 nM cycloheximide increased Arc protein within 10 min. Even at these low doses, cycloheximide effectively reduced general protein synthesis. 100 nM cyclohex-imide reduced the total incorporation of ³⁵S labeled methionine and cysteine into TCA precipitant to ˜60%. Previous studies have noted the paradoxical action of low dose cycloheximide to increase the synthesis of specific proteins, and rationalized this action by suggesting that global reduction of elongation can increase the availability of factors that are required for translation initiation of specific transcripts that are poorly initiated under control conditions.

### Example 5

### eEF2K Physically Associates with Homer and Group I mGluRs

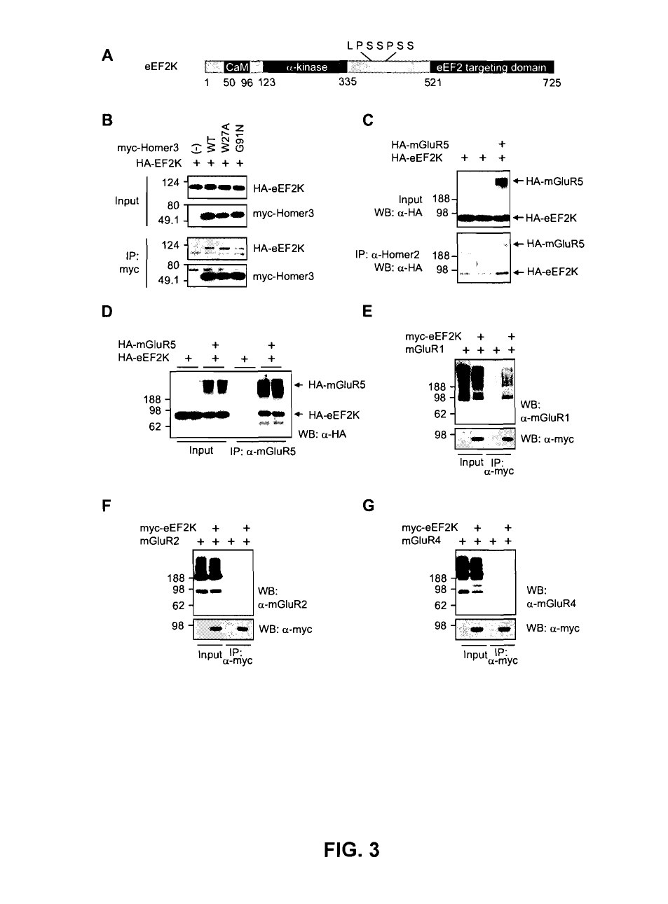

Homer proteins bind group I mGluRs and play a role in their signaling by also binding signaling partners, including IP₃R. Homer proteins bind two known sequence motifs; PPxxF (type 1) (SEQ ID NO:5) and LPSSPSS (type 2) (SEQ ID NO:6). A search for candidate Homer binding molecules (http://us.expasy.org/cgi-bin/scanprosite), revealed that eEF2K possess a type 2 Homer binding motif (FIG. 3A). eEF2K is a highly conserved enzyme that phosphorylates and regulates its only known substrate, eEF2. The N-terminal half of eEF2K contains a Ca²⁺-calmodulin (CaM) binding site which is required for its activation, and an α-kinase domain. The C-terminal half of eEF2K functions as a targeting domain that is required for it to phosphorylate eEF2. A linker region between the N- and C-terminus includes the putative Homer binding site, and is phosphorylated by multiple signaling kinases including PI3K/mTOR/S6K, MAPK, and PKA.

eEF2K and Homer were co-immunoprecipitated (co-IP) from HEK293T cells (FIG. 3B). The EVH1 domain of Homer is required to bind eEF2K, and mutation of a critical binding surface for polyproline ligands [Homer3 G91N] disrupted binding. As anticipated by conservation of their EVH1 domains, Homer 1, 2 and 3 bind eEF2K (not shown).

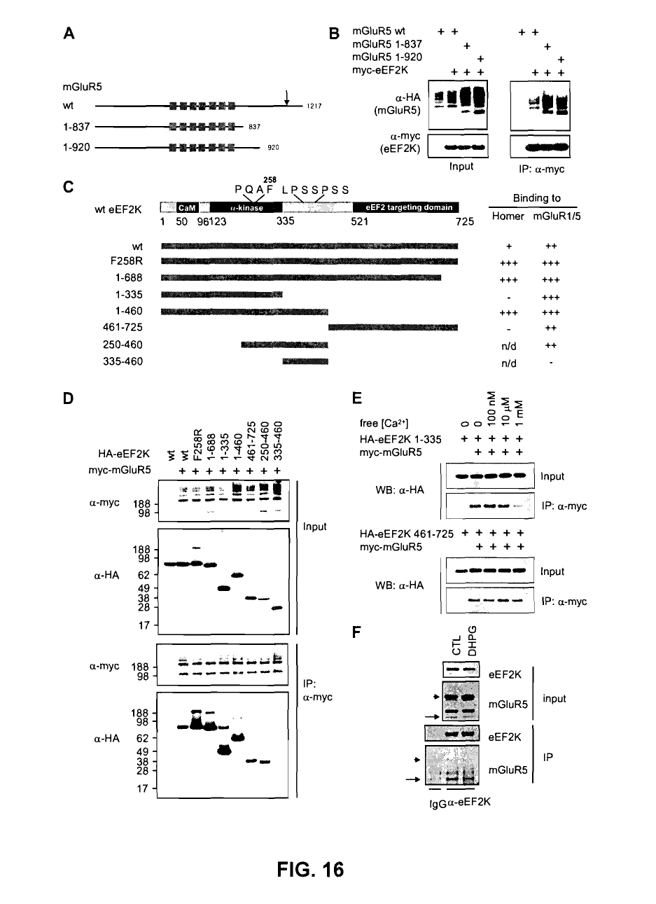

Conditions that might regulate Homer-eEF2K binding were examined and it was found that co-expression of mGluR5 strongly enhanced binding (FIG. 3C). Moreover, eEF2K was found to interact with group I mGluRs independently of Homer. The interaction of eEF2K and group I mGluRs was observed even when Homer was not co-expressed (FIGS. 3D and 3E), and eEF2K bound to mGluR5 mutants that do not bind Homer (FIGS. 16A and 16B). eEF2K also co-Wed with mGluR1 (FIG. 3E), another member of group I mGluRs, but not with other mGluRs including mGluR2 and mGluR4 (FIGS. 3F and 3G).

To identify regions of eEF2K that are critical for binding Homer and mGluR, a deletion analysis of eEF2K was performed (FIGS. 16C and 16D). The linker region of eEF2K, which includes the type 2 Homer ligand, appears essential for binding Homer since N-terminal fragments that include this region bind, while C-terminal fragments or N-terminal fragments that do not include the linker region, do not bind. eEF2K binding to mGluR5 appears more complex since both N- and C-terminal fragments of eEF2K bind mGluR5 (FIG. 16C). These data suggest that mGluR, Homer and eEF2K assemble by multiple interactions into a tertiary complex.

FIG. 3 shows eEF2K binds Homer and mGluR1/5; (A) Schematic diagram of eEF2K. The N-terminus of eEF2K contains a Ca²⁺/calmodulin (CaM) binding motif and an α-kinase domain. The C-terminal eEF2 targeting domain, which recruits the substrate, eEF2, is linked to the hyperphosphorylated internal region. Putative Homer binding site is shown above the diagram. (B) Co-immunoprecipitation (co-IP) of eEF2K and Homer. HA-tagged (HA-) eEF2K was co-expressed with myc-tagged WT, W27A, or G91N Homer3 in HEK293T cells and IP was performed with anti-myc antibody. HA-eEF2K co-IPed with WT or W27A Homer 3 was co-expressed but not with G91N Homer. (C) mGluR5 increases the interaction of eEF2K and Homer. HA-eEF2K was transfected with or without HA-mGluR5. IP was performed by anti-Homer2 antibody, which Wed endogenous Homer2 protein.

Western blot was performed using anti-HA antibody. Co-IP of HA-eEF2K was increased when mGluR5 was co-expressed. (D) eEF2K co-IPs with mGluR5. HEK293T cells were transfected with HA-eEF2K with or without HA-mGluR5 and lysates were IPed with anti-mGluR5 antibody and blotted with anti-HA antibody. eEF2K co-IPed only when mGluR was co-expressed. Samples were boiled before loading to aggregate and separate mGluR5 monomer from eEF2K. (E) mGluR1 co-IPs with eEF2K. HEK293T cells were transfected with mGluR1 and eEF2K, and lysates were Wed with mycAb. Samples were not boiled to show the monomer of mGluRs. (F and G) mGluR2 and mGluR4 do not co-IP with eEF2K.

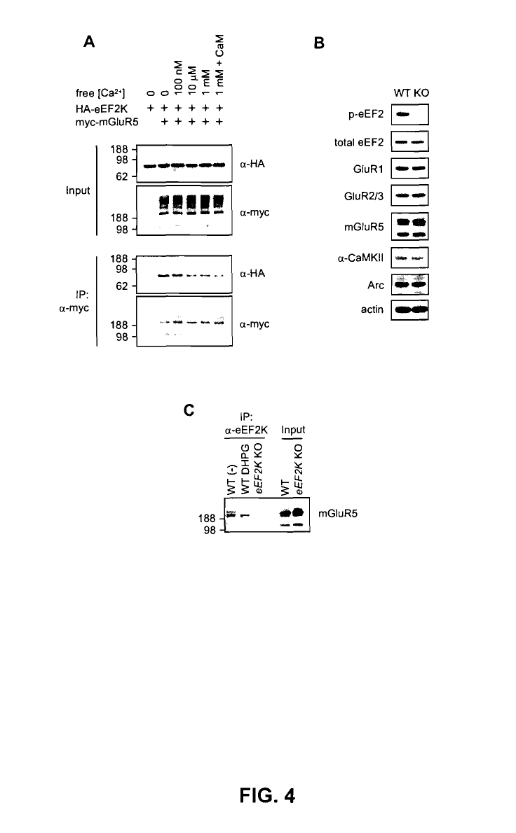

FIG. 16 shows Analysis of eEF2K interaction with Homer and mGluR5; (A and B) The C-terminal cytoplasmic tail of mGluR5 is not required for co-IP with eEF2K. Indicated constructs were co-expressed in HEK293 cells and assayed for co-IP. The arrow indicates the Homer binding site on mGluR5. Gray boxes indicate transmembrane domains. (C and D) Schematic diagram of eEF2K deletion mutants. Mutants were expressed in HEK293T cells and assayed for co-IP with native Homer or with co-expressed myc-mGluR1 or mGluR5. Data for co-IP with mGluR5 is shown in D. mGluR5 co-IPed the N-terminal and C-terminal fragments of eEF2K but not the middle part of eEF2K (aa335-460). Point mutation in the α-kinase domain (F258R) and small deletion of C-terminal part (aa1-688) robustly enhanced the binding. IP was performed as indicated in FIG. 3. (E) High concentration of free calcium inhibits binding of mGluR5 to an N-terminal fragment of eEF2K but not to a C-terminal fragment of eEF2K that lacks the CaM binding domain. IP was performed as indicated in FIG. 4A. (F) Cultured neurons were stimulated by DHPG (100 μM, 20 min) and co-IP was performed with an anti-eEF2K antibody. mGluR5 was co-immunoprecipitated with eEF2K but not by control IgG Stimulation of group I mGluRs with DHPG decreased the interaction of mGluR5 and eEF2K. Arrow and arrowhead mark monomer and dimer forms of mGluR5.

### Example 6

### The Interaction of eEF2K with mGluR is Dynamic and is Modulated by Ca²⁺ and mGluR Activity

The kinase activity of eEF2K is known to be regulated by Ca²⁺ via its Ca²⁺-CaM binding domain (Nairn et al., J Biol Chem., 262, 17299-17303 (1987) and Ryazanov, et al., FEBS Lett., 214, 331-334(1987)). To test whether Ca²⁺ modulates the mGluR5-eEF2K binding, co-IP experiments were performed using lysates from co-transfected HEK293T cells in the presence of defined concentrations of free Ca²⁺ (FIG. 4A). Co-IP was robust at [Ca²⁺] less than 1 μM but markedly decreased at concentrations >10 μM. mGluR5 binding to a C-terminal fragment of eEF2K that lacks the CaM binding domain but retains binding to mGluR5 was not inhibited by [Ca²⁺] (FIG. 16E). These results indicate that [Ca²⁺] can modulate the interaction of group I mGluRs with eEF2K, and suggest a role for CaM binding to eEF2K.

eEF2K KO mice were used for the analysis of mGluR-eEF2K binding. eEF2K KO mice were viable and fertile and showed the anticipated absence of phosphorylated eEF2 (Thr56) (FIG. 4B). The levels of several synaptic proteins were not altered in the hippocampus of KO mice (FIG. 4B). Synaptoneurosomes from fore-brains of WT and eEF2K KO mice were prepared and stimulated with DHPG for 20 min. Co-IP experiments using anti-eEF2K antibody confirmed that native mGluR5 associated with eEF2K (FIG. 4C). The co-IP of mGluR5 was reduced when synaptoneurosomes were stimulated with DHPG. Interaction of endogenous mGluR5 and eEF2K was also reduced upon DHPG stimulation of cultured neurons (FIG. 16F). This demonstrates that mGluR and eEF2K associate in vivo, and their interaction is reduced by mGluR activation.

FIG. 4 shows dynamic interaction of eEF2K and mGluR5; (A) Calcium dissociates eEF2K from mGluR5. HEK293T cells were transfected with HA-eEF2K with or without myc-mGluR5 and cells were harvested with lysis buffer without calcium or containing various concentrations of free calcium. Calmodulin (CaM) (25 μg/ml) was also added to the lysis buffer as indicated. Binding was decreased at [Ca⁺] higher than 10 μM. (B) Phospho-eEF2 was not detected in the hippocampus of eEF2K KO, while the level of total eEF2, GluR1, Glur2/3, mGluR5, α-CaMKII, Arc, and actin was not altered in eEF2K KO mice compared to WT littermate controls. (C) Synaptoneurosomes, prepared from the forebrain of eEF2K KO and WT mice, were stimulated with vehicle or DHPG for 20 min. Synaptoneurosomes were then lysed and immunoprecipitated with anti-eEF2K antibody. mGluR5 co-IPed with eEF2K only in WT samples. Stimulation of synaptoneurosomes with DHPG decreased the co-IP of mGluR5.

### Example 7

### Group I mGluRs Dynamically Regulate the Phosphorylation of eEF2

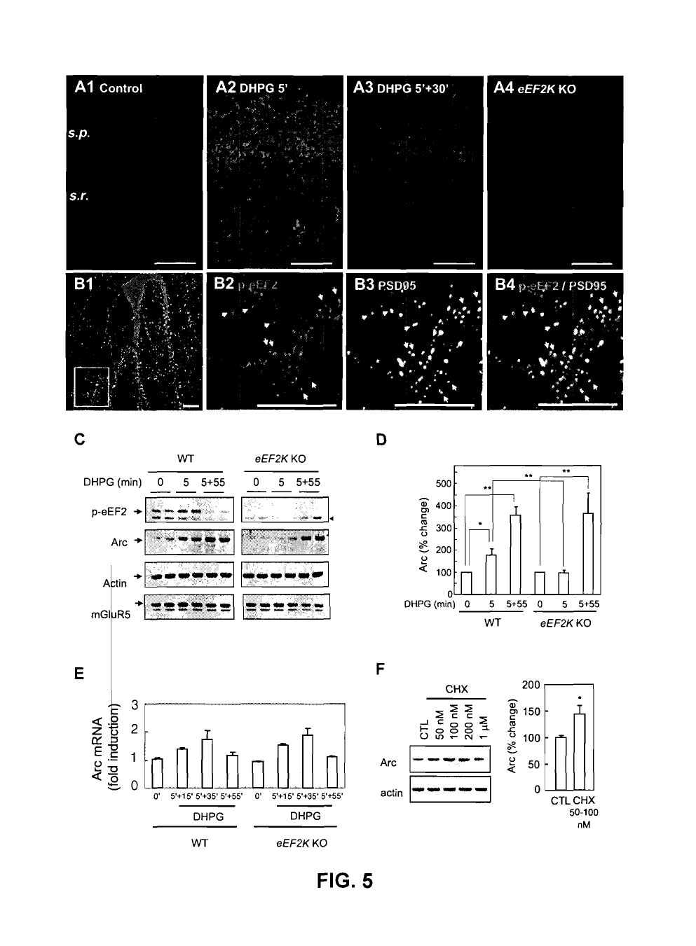

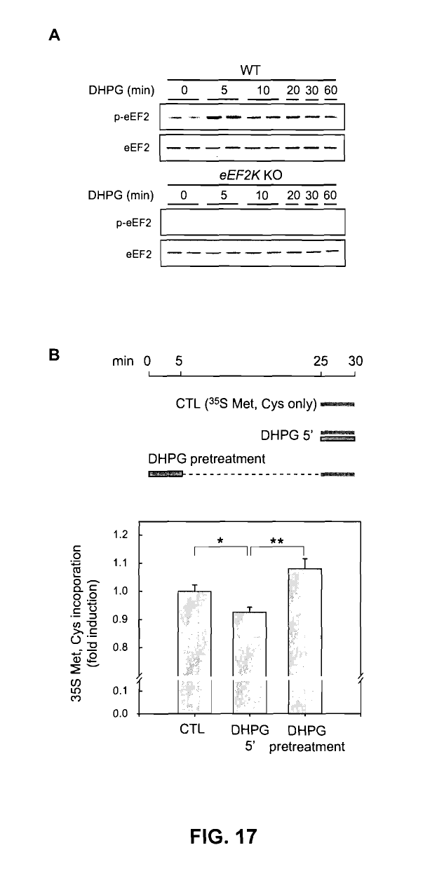

Activated eEF2K selectively phosphorylates eEF2. To assess whether mGluR activates this pathway in conditions that evoke LTD, the level of phospho-eEF2 in hippocampal slices of either WT or eEF2K KO mice was monitored using the same stimulus parameters that induce mGluR-LTD. Activation of mGluR increased the phosphorylation of eEF2 in the stratum pyramidal (s.p.), and stratum radiatum (s.r) of the hippocampal CA1 region within 5 min (FIG. 5A). By 30 min after washout of DHPG, the level of phospho-eEF2 was reduced to pre-stimulation level. No phosphorylation of eEF2 was detected in eEF2K KO slices. The transient induction of phospho-eEF2 by DHPG was confirmed by western blot analysis in hippocampal slices (FIG. 17A).

To further examine dendritic localization of eEF2K activity, DIV14 neurons were stimulated with DHPG for 5 min and stained with phospho-eEF2 and PSD95, a marker for excitatory synapses (FIG. 5B). Phospho-eEF2 showed a distinct punctal distribution in spines that co-localized with PSD95. Phospho-eEF2 was also present in dendritic shafts and the cell body. Staining was absent in eEF2K KO cultures (data not shown). This result is consistent with a previous report that translational regulators, including eEF2K, are enriched in synaptic fraction.

Phosphorylation of eEF2 is known to inhibit translational elongation. Therefore, the prediction that global protein translation might be transiently reduced co-incident with the transient increase of phospho-eEF2 was examined. Stimulation of neurons with DHPG for 5 min transiently decreased the incorporation of ³⁵S amino acids into TCA precipitants, and this effect was reversed 20 min after washout of DHPG (FIG. 17B). A previous study reported that DHPG rapidly increased protein synthesis in synaptoneuronsomes (Weiler, et al., PNAS USA, 101, 17504-17509(2004)). DHPG did not induce p-eEF2 in synaptoneuronsomes (data not shown), and it is possible that the eEF2 dependent translational mechanism is not maintained in broken cell preparations.

FIG. 5 shows rapid induction of Arc by group I mGluRs dependent on eEF2K. (A) Hippocampal slices were prepared from WT and eEF2K KO mice and were stimulated with DHPG for 5 min. phospho-eEF2 (p-eEF2, red) in area CA1 was increased by DHPG within 5 min and declined by 30 min following washout. Specificity of phospho-eEF2 was confirmed by staining of eEF2K KO slices. s.p., stratum pyramid-dal; s. r., stratum radiatum (B) Cultured hippocampal neurons were treated with DHPG for 5 min and were stained with phospho-eEF2 (red) and PSD95 (green) antibodies on DIV14. Phospho-eEF2 showed punctal distribution in dendritic spines and dendritic shafts. Phospho-eEF2 in spines colocalized with PSD95 (arrows). B2, B3, B4 are enlarged images of the rectangular region of B1. (C and D) mGluR-dependent rapid synthesis of Arc is absent in eEF2K KO neurons. Neurons from the forebrains of WT or eEF2K KO mice were cultured for DIV14 and treated with DHPG (50 μM, 5 min). Phosphorylation of eEF2 was undetectable in eEF2K KO neurons. No difference in the level of mGluR5 was observed between WT and eEF2K KO neurons. An arrow head indicates a non-specific band. P-values were obtained by paired t-test comparing basal and drug-treated levels. P-values for comparison of WT and eEF2K KO mice were obtained by Student's t-test. * p<0.05, ** p<0.01, n=8. Error bars are S.E.M. (E) Arc mRNA express-ion is not altered in eEF2K KO neurons. The level of Arc mRNA was measured in WT and eEF2K KO neurons following the stimulation with DHPG. (F) Low dose cyclohex-imide (CHX) increases Arc protein expression. Cultured eEF2K KO neurons were treated with indicated doses of CHX for 10 min. * p<0.05, n=8.

FIG. 17 shows eEF2K activity regulated by group I mGluRs; (A) Western blot analysis of phospho-eEF2 in the hippocampal slices of WT and eEF2K KO mice. DHPG (50 μM, 5 min) dramatically increased the phospho-eEF2. After 5 min of treatment, DHPG was washed out and slices were kept in the artificial cerebrospinal fluid (ACSF) until the time indicated. The level of phospho-eEF2 returned to the baseline after wash out. (B) Global protein synthesis was monitored by measuring the incorporation of ³⁵S-labeled methionine and cysteine into TCA precipitant. Radiolabelled amino acids were added to regular neuronal culture medium for 5 min (red line, bar 1). Simultaneous treatment of DHPG for 5 min (gray box) decreased global protein synthesis (0.926±0.017 compared to untreated control, bar 2). However, pretreatment of DHPG (30 min before harvest for 5 min) increased global protein synthesis (1.080±0.036 compared to untreated control, bar 3). P-values are shown on top of the bars. * p<0.05, ** p<0.01, n=4

### Example 8

### Rapid De Novo Arc Translation is Selectively Absent in eEF2K KO Neurons

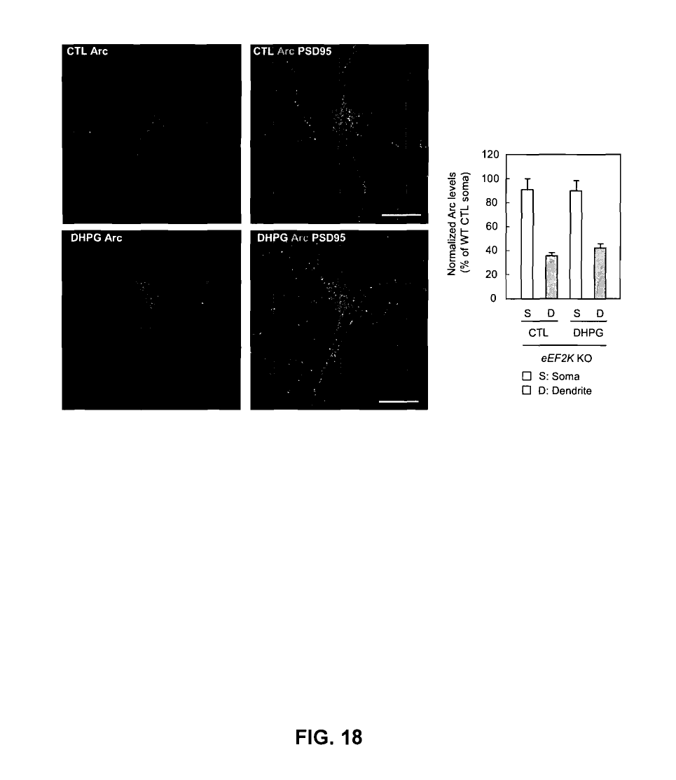

Arc expression was examined in DIV14 forebrain neuronal cultures prepared from WT and eEF2K KO mice. The steady state expression of Arc protein was identical in WT and eEF2K KO neurons, however the increase in Arc protein 5 min after DHPG in WT neurons was absent in eEF2K KO neurons in both biochemical (FIGS. 5C and 5D) and immunocytochemical assays (FIG. 18). By contrast, Arc protein was induced to the same extent in WT and eEF2K KO neurons 60 min after DHPG stimulation. Arc mRNA was identical in WT and eEF2K KO neurons prior to application of DHPG, and increased identically at 40 min after stimulation in both WT and eEF2K KO neurons (FIG. 5E). Accordingly, the lack of rapid induction of Arc protein in the eEF2K KO neurons is not due to reduced Arc mRNA expression. mGluR signaling that is required for induction of Arc mRNA and the delayed increase of Arc protein are intact in eEF2K KO neurons. Moreover, Arc protein expression is identical in hippocampus of WT and eEF2K KO mice (FIG. 4B) indicating that eEF2K is not required for basal expression of Arc protein in vivo.

If the failure of DHPG to induce rapid synthesis of Arc protein in the eEF2K KO neurons is due to a selective interruption of the action of phospho-eEF2, then the low dose of cycloheximide, which does not require eEF2K or phopho-eEF2 to inhibit the elongation step, should induce the synthesis of Arc protein in eEF2K KO neurons. Treatment of DIV14 eEF2K KO neurons with low dose cycloheximide (50 nM and 100 nM) increased the level of Arc protein in eEF2K KO neurons (FIG. 5F), similar to WT neurons (FIG. 2D). High dose cycloheximide (>1 uM) did not induce Arc in either WT and eEF2K KO neurons. The ability of low dose cycloheximide to rescue rapid Arc induction indicates that mechanisms that mediate rapid Arc translation subsequent to inhibition of elongation are intact in eEF2K KO neurons.

FIG. 18 shows rapid induction of Arc protein by DHPG absent in eEF2K KO hippocampal neurons; The level of Arc was monitored by immunohistochemistry of cultured hippocampal neurons derived from eEF2K KO mice as shown in FIG. 2A. DHPG did not change the level of Arc in either the soma or dendrites. Green, red, and blue colors show PSD95, Arc, and DAPI, respectively.

### Example 9

### mGluR-LTD and PP-LFS LTD are Selectively Absent in eEF2K KO Hippocampal Slices

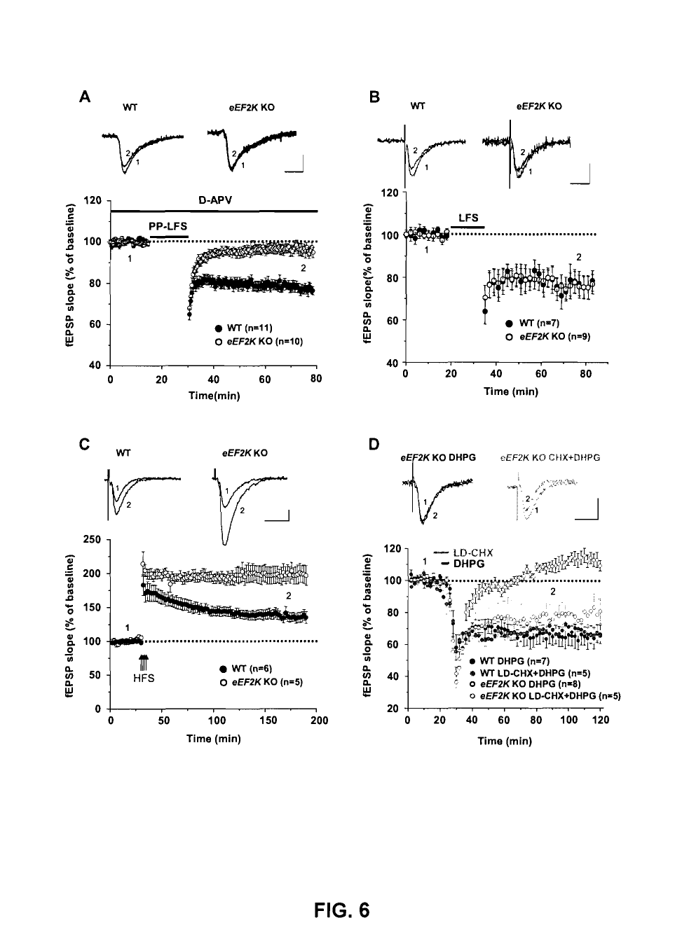

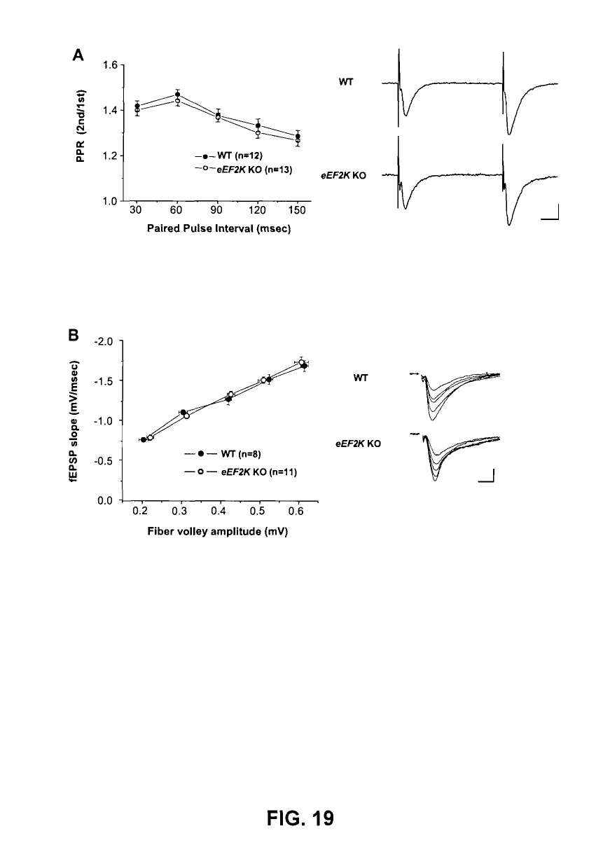

The role of eEF2K in plasticity of the Schaffer collateral-CA1 synapse was tested using acute hippocampal slices. Baseline measures of synaptic strength and presynaptic function were not altered in the eEF2K KO slices (FIG. 19). However, LTD induced by either PP-LFS (97.5±2.4% of baseline) or DHPG (108.7±3.6% of baseline) was impaired in the eEF2K KO slices (FIGS. 6A and 6D). The immediate short-term synaptic depression following DHPO stimulation was identical in WT and eEF2K KO slices, however, synaptic strength returned to near baseline levels in the eEF2K KO slices. Similarly, synaptic transmission returned to near baseline levels within 10 min of completion of the PP-LFS protocol.

In contrast to the marked deficit of mGluR-dependent LTD, NMDAR-dependent LTD was identical in time course and stability in slices derived from eEF2K KO mice (72.7±2.2% of baseline) compared to WT mice (73.1±3.4% of baseline) (FIG. 6B). LTP was also preserved (FIG. 6C). LTP of Schaffer collateral-CA1 synapses was induced by four trains of high frequency stimulation with an intertrain interval of 3 s. In WT slices, fEPSP was increased to 171.5±13.4% of baseline immediately after stimulation (t=30 min) and sustained at the level of 138.4±7.7% of baseline at t=175 min.

These stimulus parameters are reported to evoke a form of synaptic plasticity that requires de novo protein synthesis for maintenance longer than ˜60 min and is referred to as late LTP (L-LTP). In slices prepared from eEF2K KO mice, the initial induction was 204.6±8.9% of baseline at t=30 min and this was sustained for 3 hours after stimulation (200.1±11.9% of baseline at t=175 min) (FIG. 6C). The magnitude of LTP was signifcantly greater in eEF2K KO than WT after 30 min of induction (p<0.005). These results indicate that eEF2K KO disrupts mGluR-LTD, but does not alter NMDAR-dependent LTD or early LTP. The apparent enhancement of late phase LTP deserves further study.

The proposed mechanism for the mGluR-LTD deficit in the eEF2K KO slices is linked to failure to rapidly translate Arc. Since low dose cycloheximide induced Arc synthesis and did not depend on phospho-eEF2, the possibility that cycloheximide could rescue mGluR-LTD in slices from eEF2K KO mice was examined. A 10 min exposure to 50-75 nM cycloheximide (low dose CHX: LD-CHX) beginning 5 min prior to addition of DHPG rescued mGluR-dependent LTD in the eEF2K KO slice (75.7±7.4% of baseline p<0.001 compared to DHPG only in eEF2K KO slices) (FIG. 6D). The same treatment of WT slices did not substantially alter the time course of mGluR-LTD (69.0±2.6% of baseline p>0.5 compared to DHPG only in WT slices). Low dose cycloheximide had no effect on baseline synaptic transmission in the absence of mGluR stimulation (101.2±2.0% for WT slices; 100.4±4.6% for eEF2K KO slices). These observations confirm that mGluR signaling required for mGluR-LTD is selectively impaired in eEF2K KO in a manner that can be rescued by transient application of low dose cycloheximide.

FIG. 6 shows mGluR-LTD impaired in hippocampal slices derived from eEF2K KO mice; fEPSPs were recorded in the hippocampal CA1 region of slices derived from eEF2K KO mice and compared to WT littermate controls. (A) Time course of the change in fEPSP slope produced by paired pulse low frequency stimulation (PP-LFS: at 1 Hz, 50 msec interstimulus interval, for 15 min) in the presence of D-APV (50 μM). LTD of WT mice was 77.0±2.1% of baseline at t=75 min (n=13). In eEF2K KO mice, fEPSPs were 97.5±2.4% of baseline t=75 min (n=15) (p<0.0001). (B) Time course of the change in fEPSP slope by low frequency stimulation (LFS: 1 Hz for 15 min).

This form of NMDAR-dependent LTD was not altered in eEF2K KO hippocampal slices (72.7±2.2% of baseline at t=75 min, n=9) compared to WT (73.1±3.4% of baseline at t=75 min, n=7) (p>0.5). (C) Late-phase of LTP was induced by 4 stimulus trains (100 Hz each) with an intertrain interval of 3 s. In WT, fEPSPs were increased to 171.5±13.4% of baseline immediately after stimulation (t=30 min) and were sustained at the level of 138.4±7.7% of baseline at t=175 min (n=6). However, in eEF2K KO, the initial LTP (204.6±8.9% of baseline at t=30 min) was maintained for 3 hours after stimulation (200.1±11.9% of baseline at t=175 min, n=5).

LTP was significantly greater in slices derived from eEF2K KO mice compared to those from WT mice at this time point (p<0.005). (D) Average time course of the change in fEPSP slope induced by DHPG (50 μM, for 5 min). LTD of WT mice was 64.7±5.2% of baseline at t=90 min (n=7). In eEF2K KO mice, LTD was significantly impair-ed (108.7±3.6% of baseline at t=90 min, n=8). Treatment with low dose cycloheximide (LD-CHX, 50-75 nM) for 10 min starting from 5 min prior to DHPG restored DHPG-LTD in eEF2K KO (75.7±7.4%, n=5). In WT mice, treatment with LD-CHX did not alter the expression of LTD (69.0±2.6%, n=5). p<0.001 when eEF2K KO DHPG only was compared to eEF2K KO LD-CHX+DHPG, WT DHPG only or WT LD-CHX+DHPG. Scale bars=0.5 mV/10 ms.

FIG. 19 shows characterization of Schaffer collateral-CA1 synapses of eEF2K KO. fEPSPs measured in the Schaffer collateral-CA1 synapses of eEF2K KO mice and compared to WT littermate controls; (A) Relationship between paired-pulse interval and PPR of the Schaffer collateral-CA1 synapses of eEF2K KO and WT slices. (B) Relationship between fiber volley amplitude and fEPSP slope of the Schaffer collateral-CA1 synapses of eEF2K KO and WT slices. Scale bars=0.5 mV/10 ms.

### Example 10

### mGluR-LTD, but Not Homeostatic Plasticity, is Disrupted in eEF2K KO Neurons in Culture

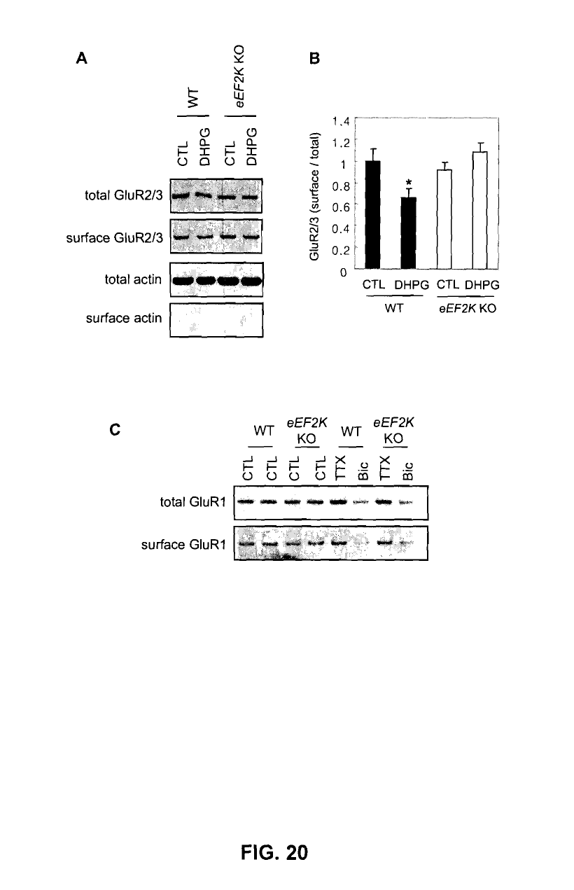

To further assess the selectivity of the eEF2K KO effect on neuronal function, two forms of neuronal plasticity that can be assayed in primary neuronal cultures were examined. Treatment of cultures with DHPG for 5 min to evoke mGluR-LTD reduced the ratio of surface to total GluR2/3 by ˜30% in WT neurons, but did not significantly reduce this measure in eEF2K KO neurons (FIGS. 20A and B). This result parallels the deficit of mGluR-LTD seen in acute slices. Cultures were also assayed for homeostatic adaptations of surface AMPA receptors since this response is markedly altered in Arc KO neurons. Treatment of eEF2K KO cortical cultures for 2 days with either tetrodotoxin (TTX) or bicuculline evoked homeostatic increases and decreases of surface GluR1 that were identical to WT neurons (FIG. 20C). Thus eEF2K KO results in a selective disruption of mGluR-dependent LTD.

FIG. 20 shows reduction of surface AMPAR by mGluR stimulation absent in eEF2K KO cultured neurons; and (A) Representative blot of surface biotinylated GluR2/3 from WT and eEF2K neurons. Stimulation of group I mGluRs with DHPG (50 μM, 5 min stimulation followed by 55 min incubation in the original medium) reduced the ratio of surface/total level of GluR2/3 in WT cultures but not in eEF2K KO cultures. (B) Surface GluR2/3 was significantly reduced 60 min after stimulation with DHPG (n=6, * p<0.05). (C) Homeostatic adaptation of surface AMPA receptor was intact in eEF2K KO neurons. Neurons were treated with tetrodotoxin (TTX:1 μM) or bicuculline (Bic: 40 μM) for 2 days and the surface expression of GluR1 subunit of AMPA receptors was measured by surface biotinylation assay. Chronic network inactivity by TTX increased the surface expression of GluR1 both in WT and eEF2K KO neurons. Surface expression of GluR1 was equally decreased by Bic both in WT and eEF2K KO neurons.

### Example 11

### Fmr1 KO disrupts Rapid, but not Delayed Induction of Arc Protein

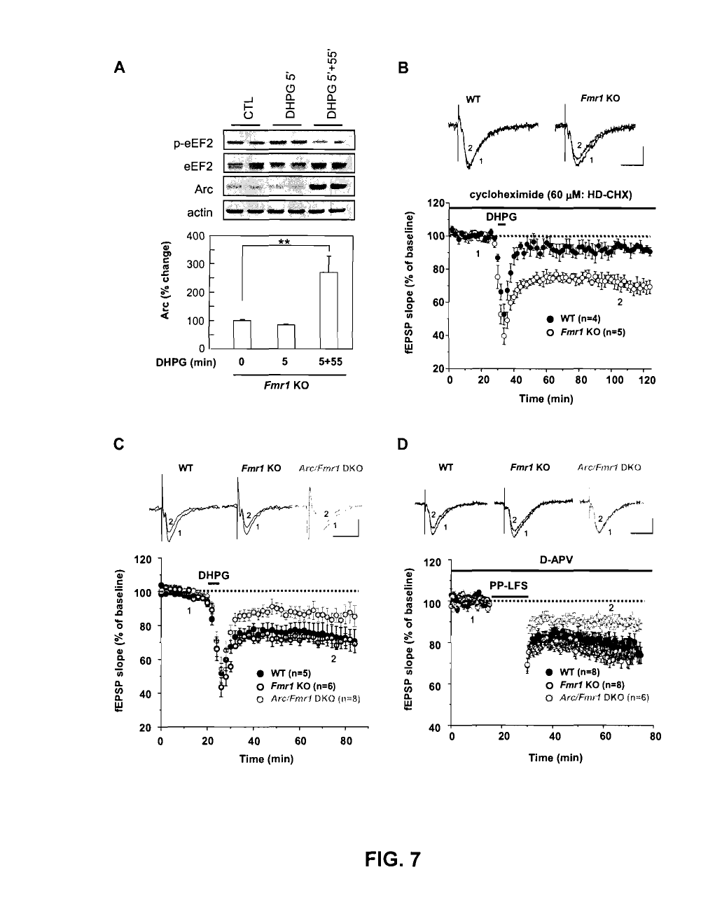

The role of the eEF2K/eEF2/Arc mechanism in the aberrant plasticity described in Fmr1 KO mice was examined. FMRP binds Arc mRNA and is hypothesized to inhibit translation prior to mGluR-stimulation. To assess whether FMRP might be critical for either rapid or delayed induction of Arc protein following mGluR stimulation, primary neuronal cultures from Fmr1 KO mice were prepared and stimulated with DHPG. Arc expression in unstimulated cultures was not consistently different between WT and Fmr1 KO neurons. Moreover, Arc protein increased 60 min after DHPG stimulation in Fmr1 KO neurons identically as in WT neurons (FIG. 7A). However, the rapid increase of Arc protein following DHPG stimulation was absent in Fmr1 KO neurons (FIG. 7A). DHPG activated mGluR/eEF2K signaling in the Fmr1 KO neurons since phospho-eEF2 was identically induced as in WT neurons (FIG. 7A).

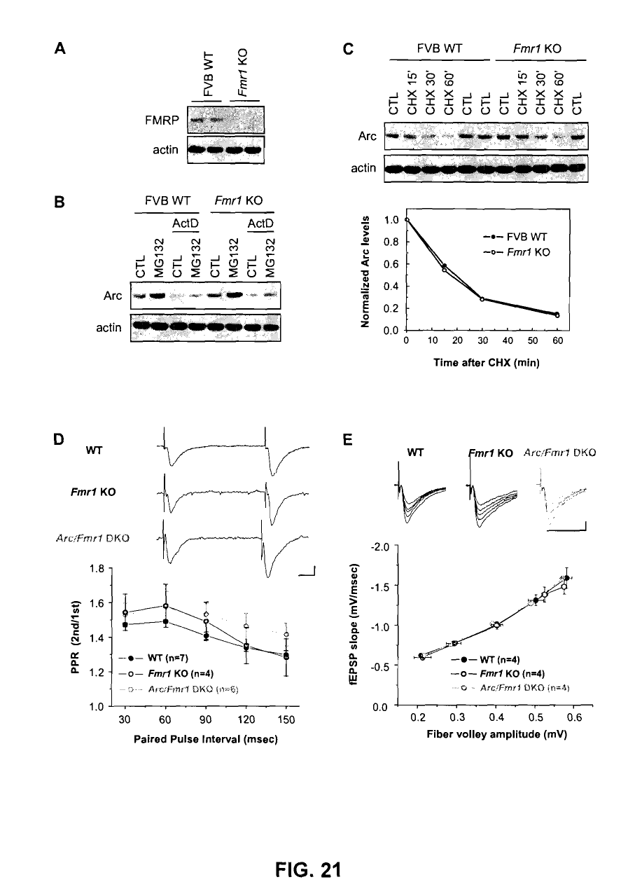

Assays of Arc protein stability and induction following proteasome inhibition with MG132 did not reveal differences between WT and Fmr1 KO neurons (FIGS. 21B and 21C). Biochemical experiments to monitor Arc expression using acute hippocampal slices revealed that basal Arc expression was highly variable even when normalized to total protein or actin, indicating a limitation of this preparation. When examined histochemically, basal Arc varied through the thickness of the slice (data not shown). Fmr1 KO neurons selectively lack the ability to rapidly up-regulate Arc expression. The reported increased of Arc mRNA in polysome fractions from Fmr1 KO mice, suggests that failure to detect a DHPG-evoked rapid increase of Arc protein is linked to elevated constitutive expression.

FIG. 7 shows LTD impaired in hippocampal slices derived from Arc/Fmr1 double KO mice; (A) DIV14 Fmr1 KO neurons were treated with DHPG as indicated in FIG. 5C. Rapid synthesis, but not delayed synthesis of Arc, was absent in Fmr1 KO. The regulation of phospho-eEF2 was intact in Fmr1 KO neurons. (B) High dose cycloheximide (60 μM: HD-CHX) did not block DHPG-LTD of Fmr1 KO slices. In the presence of high dose of cycloheximide, DHPG-LTD of Fmr1 KO was 72.3±4.8% of baseline at t=105 min (n=5), while DHPG-LTD in WT (FVB) slices was blocked (fEPSP was 95.5±2.9% of baseline at t=105 min (n=4); p<0.01 when Fmr1 KO was compared to FVB WT.) (C) Average time course of fEPSP slope of Arc/Fmr1 double KO (DKO) mice. mGluR-LTD was induced by DHPG (50 μM, for 5 min).

DHPG-LTD of Arc/Fmr1 DKO was 85.9±4.1% of baseline at t=75 min (n=8). In Fmr1 KO, DHPG-LTD was 68.2±2.6% of baseline at t=75 min (n=6). In WT, DHPG-LTD was 73.0±6.6% of baseline at t=75 min (n=5). p<0.01 when Arc/Fmr1 DKO was compared to either WT or Fmr1 KO. fEPSPs of post-DHPG in Fmr1 KO were not significantly different from those in FVB WT. (D) Time course of the change in fEPSP slope by PP-LFS. PP-LFS LTD of Arc/Fmr1 DKO was 88.3±2.1% of baseline at t=65 min (n=6). In Fmr1 KO, PP-LFS LTD was 75.5±3.7% of baseline at t=65 min (n=8). In FVB WT, PP-LFS LTD was 80.5±2.6% of baseline at t=65 min (n=8). p<0.05 when Arc/Fmr1 DKO was compared to either WT or Fmr1 KO. fEPSPs of post-DHPG in Fmr1 KO were not significantly different from those in FVB WT (p=0.4). Scale bars=0.5 mV/10 ms.

FIG. 21 shows Characterization of Arc protein and Schaffer collateral-CA1 synapses of Fmr1 KO. (A) Expression of FMRP protein in cultured neurons. (B) Basal synthesis of Arc protein was not detectably altered in Fmr1 KO neurons. One hour treatment of MG132 (10 μM) increased the levels of Arc. Inhibition of de novo transcription by Actinomycin D (ActD: 10 μM, 1 hr) decreased the basal levels of Arc. However, MG132 still increased the levels of Arc in the presence of ActD. No difference was observed between WT and Fmr1 KO cultures in these assays. (C) Stability of Arc protein was not altered in Fmr1 KO neurons. Time course of Arc protein was monitored following high dose cycloheximide treatment (CHX: 50 μM). Kinetics of Arc degradation were not altered in Fmr1 KO neurons (lower panel). (D) Relationship between paired-pulse interval and PPR of the Schaffer collateral-CA1 synapses of WT, Fmr1 KO, and Arc/Fmr1 DKO mice. (E) Relationship between fiber volley amplitude and fEPSP slope of the Schaffer collateral-CA1 synapses of WT, Fmr1 KO, and Arc/Fmr1 DKO. Scale bars=0.5 mV/10 ms.

### Example 12

### Arc is Required for mGluR-LTD and PP-LFS LTD in Fmr1 KO Mice

In anticipation of physiological studies to assess the role of Arc in synaptic plasticity of Fmr1 KO mice, Arc protein expression in the hippocampus was examined. Arc protein has previously been reported to be modestly up-regulated in both total brain and synaptosomal fractions of Fmr1 KO mice (Zalfa et al., Cell, 112, 317-327 (2003)). But in the present Example, Arc protein was not consistently different in the hippocampus (either in vivo or in acute slices) or cortex when care was taken to sacrifice mice without behavioral activation. Mice in which both Fmr1 (in FVB background) and Arc (in B6 background) were deleted were generated. Double Arc/Fmr1 KO (DKO) mice are viable, fertile and not different from WT mice in size or postnatal survival. Indices of basal synaptic transmission were normal in Fmr1 KO and Arc/Fmr1 DKO (FIGS. 21D and E).

As reported previously (Nosyreva et al., J. Neurophysiol., 95, 3291-3295 (2006)), DHPG evoked a sustained reduction of synaptic strength (68.2±2.6% of baseline for Fmr1 single KO slices; 73.0±6.6% of baseline for FVB WT slices, FIG. 7C). The Jackson laboratory provided Fmr1 KO mice in the FVB background, and the magnitude of LTD was not significantly different from FVB WT mice. As reported previously in studies of Fmr1 KO in the B6 back-ground, mGluR-LTD was not inhibited by high dose cycloheximide (60 μM) (FIG. 7B). In Arc/Fmr1 DKO (in B6/FVB), DHPG evoked an initial reduction of synaptic strength that was not different from WT, Arc KO or Fmr1 KO.

However, expression of DHPG-evoked LTD was significantly impaired in Arc/Fmr1 DKO (85.9±4.1%, p<0.01 compared to Fmr1 single KO or FVB WT). PP-LFS LTD was also impaired in Arc/Fmr1 DKO (88.3±2.1% of baseline for Arc/Fmr1 DKO slices; 75.5±3.7% of baseline for Fmr1 single KO, 80.5±2.6% of baseline for FVB WT slices, p<0.05 when Arc/Fmr1 DKO was compared to Fmr1 single KO or FVB WT, FIG. 7D). These results indicate that Arc is required for mGluR-LTD in both WT and Fmr1 KO neurons. Deletion of Arc does not entirely prevent DHPG or PP-LFS LTD, suggesting that additional mechanisms contribute to the aberrant LTD in Fmr1 KO mice.

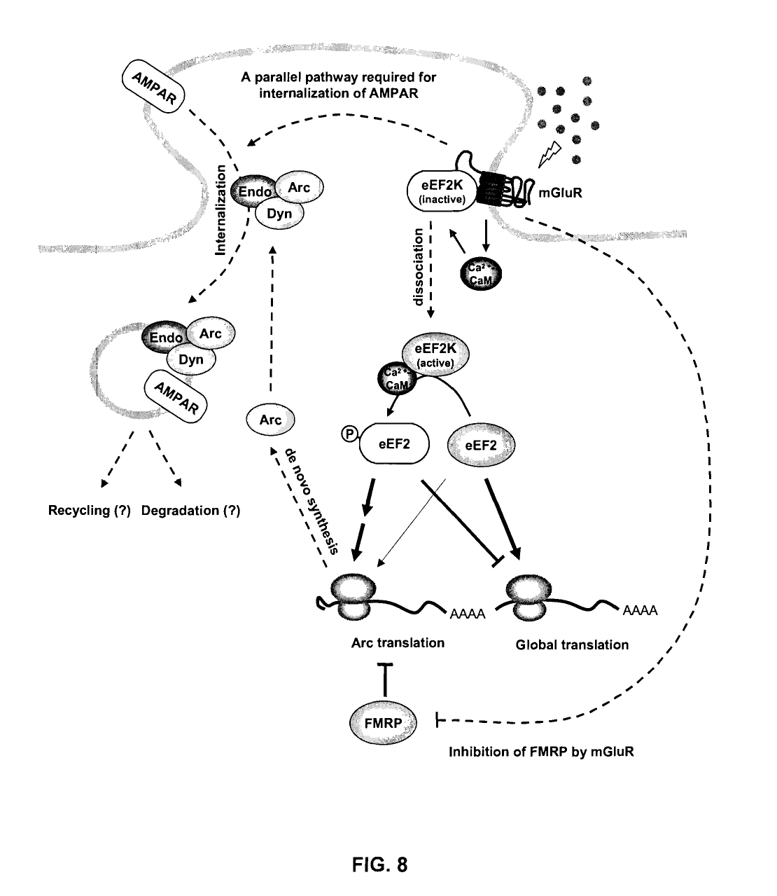

FIG. 8 shows eEF2K, FMRP and rapid, de novo translation of Arc protein in mGluR-LTD; Group I mGluRs activate eEF2K via Calcium-calmodulin (CaM). eEF2K phosphorylates eEF2, which inhibits elongation generally but increases Arc translation. Arc forms a complex with endophilin2/3 (Endo) and dynamin (Dyn) and induces the internalization of AMPAR (Chowdhury et al., 2006). FMRP inhibits the translation of Arc at the basal state. Arc induction alone is not sufficient for mGluR-LTD, indicating that mGluR activates another pathway that is required to internalize AMPAR (Cho et al., 2008). In Fmr1 KO mice, the synthesis of Arc protein is constitutively de-repressed and de novo synthesis of Arc is not required for mGluR-LTD.

### Example 13

### Reduction of Insoluble Aβ and Plaque Formation in eEF2K-KO Mice

Arc protein up-regulation has been suspected to have a role in the development of amyloid plaques in AD. The role of eEF2K in the pathogenesis of AD was therefore examined using a mouse model that expresses two human genes that are linked to familial AD; the Swedish mutation of APP (APP_{swe}), and the mutation of PS1 termed ΔE9 (PS1ΔE9). Mice that express both of these transgenes show components of AD including the deposition of insoluble Aβ and plaque formation. A eEF2K-KO mouse was crossed with APPswe/PS1ÄE9 transgenic mice and the offspring were allowed to age for 13 months, eliciting a treatment group. Specifically, 12 month-old WT background mice expressing APPswe/PS1ÄE9 were compared with 13 month-old eEF2K-KO mice also expressing APPswe/PS1ÄE9.

Whole forebrains of each group were sonicated on wet ice in 10 vol of 2% SDS. FIG. 9 shows Western blots of detergent lysates from forebrains of APPswe/PS1ÄE9 transgenic mice that are either in WT background or in eEF2K KO background. Note that proteins involved in the generation of Aβ are identical in WT and eEF2K KO mice. huAPP is human amyloid precursor protein. APP-CTFs are the C-Terminal fragments of APP that result from either alpha or β-site cleavage of APP. BACE1 is β-secretase 1, which mediates β cleavage of APP. GluR1 is the AMPA type glutamate receptor. Arc is the immediate early gene (Activity-regulated cytoskeleton-associated protein). Narp is neuronal activity-regulated pentraxin (identical to NP2). NP1 is neuronal pentraxin type 1. T-eEF2 is total eukaryotic elongation factor 2. P-eEF2 is phosphorylated eEF2. β-actin is a control for loading. mTOR is mammalian target of rapamycin. AKT is kinase. P-S6 (S240/244) and (S235/236) are phosphorylated forms of S6.

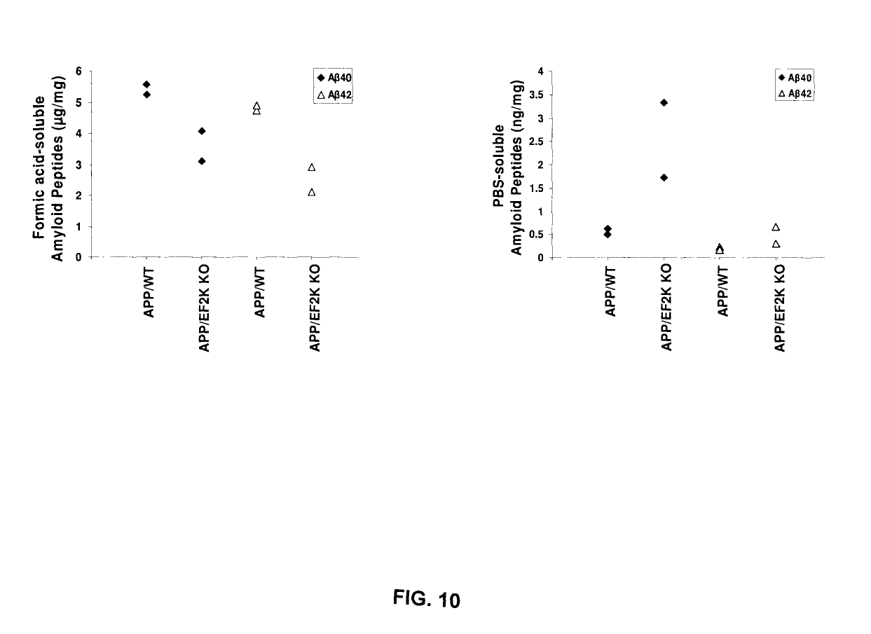

To measure the Aβ levels in vivo, the brains of APP/WT (APPSWE/PS1ΔE9; eEF2K+/+) and APP/Arc KO (APPSWERS1ΔE9; eEF2K−/−) mice were dissected on ice and homogenized in PBS buffer containing complete protease inhibitor cocktail. After the lysates were centrifuged at 100,000×g for 30 min, the supernatants containing soluble Aβ peptides were collected for assay, and the pellets were homogenized in 70% formic acid solution. After incubation on ice for 1 h, the formic acid lysates were centrifuged at 100,000×g for 1 h, and the supernatants were collected and neutralized by 1 M Tris-base solution. The concentrations of Aβ40/Aβ42 peptides in PBS-soluble fractions and formic acid-soluble fraction were measured using a quantitative sandwich ELISA kit (Biosource International) that specifically detects human Aβ40/Aβ42. BCA method was used to measure the of total protein concentrations (Pierce). FIG. 10 illustrates the results of the ELISA determination of Aβ levels in 13-month-old APP/EF2K-KO mice compared with 12-month old APP/WT mice.

Mouse brain hemispheres were then immersed in 10% formalin/PBS for histology. Brains were dehydrated in methanol, treated with xylenes and embedded in paraffin. 4 μm sagittal sections ˜800 μm from bregma were cut and used for plaque staining. Before immunostaining, slides were deparaffmized by xylenes. After rehydration through graded ethanols into water, they were incubated with 88% folic acid for 5 min. Endogenous peroxidase activity was quenched by incubation with 0.9% hydrogen peroxide in methanol. Slides were microwaved for 5 min in water, cooled gradually and washed in PBS.

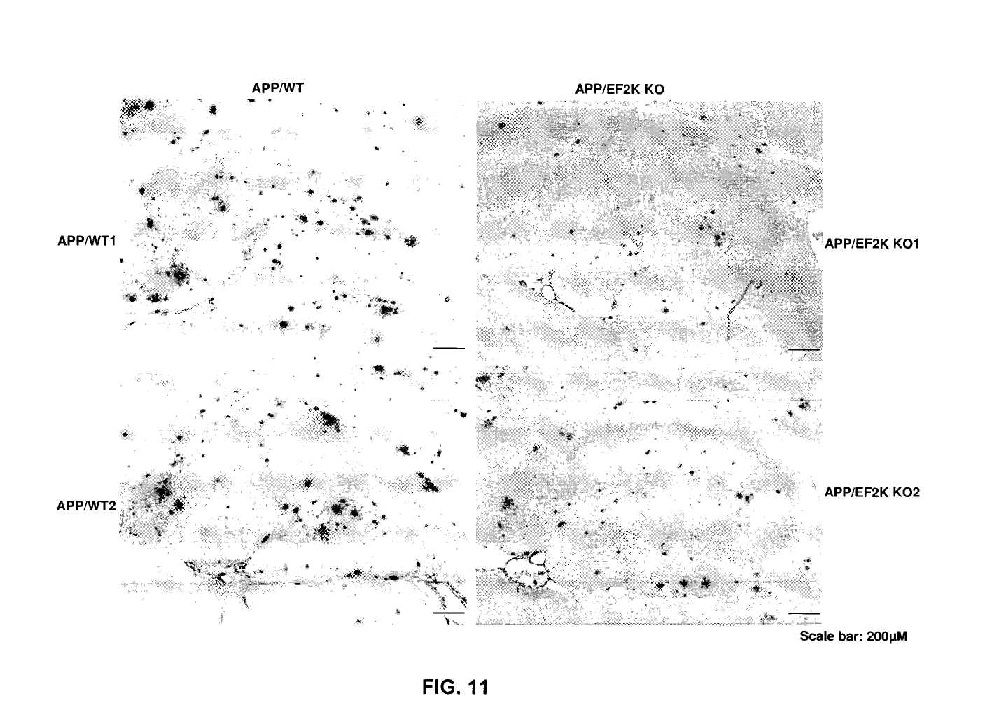

Nonspecific staining was blocked with 3% normal goat serum (NGS) in PBS for 1 hour. Slides were then incubated with anti-human Aβ antibody (6E10; 1:500 dilution) in PBS+3% NGS overnight at RT. After washing with PBS, slides were incubated with biotinylated goat anti-mouse IgG antibody (VECTOR laboratories BA-9200) in PBS+2% NGS for one hour. Then ABC reagent (VECTOR laboratories PK-6102) was applied to those sections. The sections were developed with diaminobenzidine (VECTOR laboratories SK-4100). FIG. 11 shows plaque formation in 13-month-old APP/EF2K KO mice is reduced compared to 12-month old APP/WT mice.

Quantification of plaques was carried out using Image J software (the National Institutes of Health). Pictures of 4 individual parts of cortex in each section were taken at the same condition and saved as TIFF files. To measure plaques, the background was subtracted and the same threshold was set, then the plaque area was counted automatically. All the 4 areas were summed and the percentage of plaque area was calculated to divide it by the total area. Statistic analysis was done by Mann-Whitney U test.

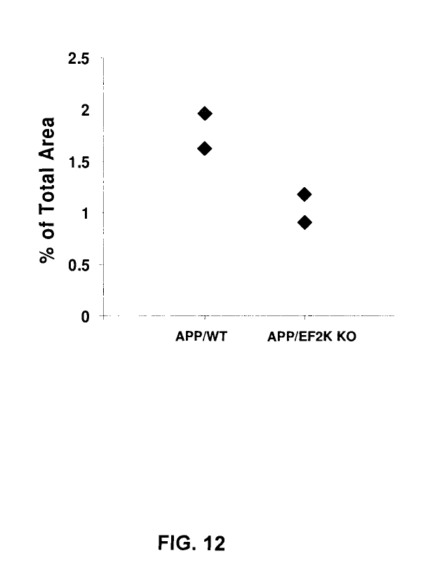

FIG. 12 shows reduction of plaque area in hippocampus of 13-month old APP/eEF2K KO vs. 12-month-old APP/WT mice. Methods for measurement of plaque area are provided above.

As shown above, substantial reductions of insoluble forms of both Aβ40 and Aβ42 in eEF2K-KO background occurred. The fact that it is reduced in the eEF2K KO is the more remarkable since amyloid deposits accelerate with age. Control studies show that the reduction of Aβ deposition is not due to changes in the amount of amyloid precursor protein (APP) or in enzymes that catalyze its cleavage including BACE1. Several synaptic proteins are also identically expressed in APPswe/PS1ΔE9 transgenic mice in WT and eEF2K KO mice.

### Example 14

### Discovery of Consensus Peptide Substrates for eEF2K

While alpha-kinase phosphorylation sites are typically found within alpha-helicies of peptide substrates, it was unknown for eEF2K if alpha-kinases recognized target substrates based on a specific primary peptide sequence around the phosphorylation site of eEF2K, or whether the alpha-helical secondary structure is responsible for the phosphorylation by alpha-kinases. To clarify this an arrayed peptide library screen was used (Turk, Yale Med. Sch. Dept. Pharmacol.) that thoroughly evaluated for specific kinase preference all 20 amino acids at each of nine positions neighboring the phosphorylation site.

Every peptide comprising this library contained a central fixed phosphorylation site where equimolar quantities of threonine and serine were introduced; each peptide also contained a carboxy-terminal biotin label. The peptide library was arrayed in a 384-well plate and consisted of twenty-two peptide mixtures in which the twenty proteogenic amino acids, phosphothreonine and phosphoserine were fixed along the peptides giving rise to a library containing 198 (22×9 a.a.) distinct peptides. Using this peptide library, kinases for various amino acids sequences were screened surrounding the phospho-acceptor site by measuring the incorporation of radiolabeled ATP for each peptide.

Reactions in this screen were run for a given incubation time and then spotted simultaneously on a streptavidin membrane through use of a high throughput capillary-based liquid transfer device. Submersion of the membrane in a specified quenching solution stripped away unincorporated ATP and then radiolabeled-ATP incorporation was measured using a phosphoimager. The quantification of ATP-incorporation for each peptide allowed the determination of which peptides were the most proficient substrates for alpha-kinases and provided an answer to whether the primary sequence or secondary structure of a substrate dictated phosphorylation by alpha-kinases.

Using this screen, it was determined that eEF2K efficiently phosphorylates pep-tides contained in this library. Preferences for certain amino acids at particular positions along the sequence were determined as well. eEF2K highly prefers basic residues at the +3 position with respect to the phospho-acceptor site. It also prefers basic and possibly serine or threonine at the +2 site. The phosphorylation motif recognized by eEF2K does not share any identity to motifs recognized by known conventional protein kinases.

The information gathered from the peptide screen assay, led to the production of the specific peptide for eEF2K, called eEF2p, that contains the consensus sequence for eEF2K phosphorylation (Ac-RKKYKFNEDTERRRFL) (SEQ ID NO: 7). In addition, a peptide with the consensus phosphorylation sequence for another alpha-kinase, TRPM7 kinase, was also generated. Both of these generated peptides have been shown to be specifically phosphorylated by their corresponding kinase in reactions carried out at a single substrate concentration (100 ìM). These newly generated peptides are considerably superior substrates than any previously identified peptides for these kinases. For example, eEF2p is two orders of magnitude more efficient that the MH-U peptide which was previously used to assay eEF2 kinase. This demonstrates that eEF2p is a highly specific substrate for eEF2 kinase. The peptide substrate can also be used for experiments on the kinetics and mode of substrate recognition for eEF2 kinase.

The development of eEF2p has allowed the development of high-throughput screening (HTS) for identification of inhibitors of eEF2 kinase. The eEF2 kinase can be produced in large quantities by E. coli and has been shown to be very stable and reactive making it an ideal source for the HTS. A HTS screen for eEF2 kinase inhibitors was developed based on the depletion of ATP by active kinase and is quantitated by coupling it with a luciferase luminescence assay, since the luciferase is ATP-dependent. Inhibition of eEF2 kinase prevented depletion of ATP that was detected as increased luminescence. eEF2K inhibitory compounds for use in the present invention may thus be identified using the HTS assay discussed herein and disclosed within U.S. Provisional Application No. 61/225,875, filed Jul. 15, 2009, the contents of which are incorporated herein by reference.

Using the HTS screen, two novel inhibitors for eEF2 kinase were identified and labeled L-587 and L-207 and have the following structure:

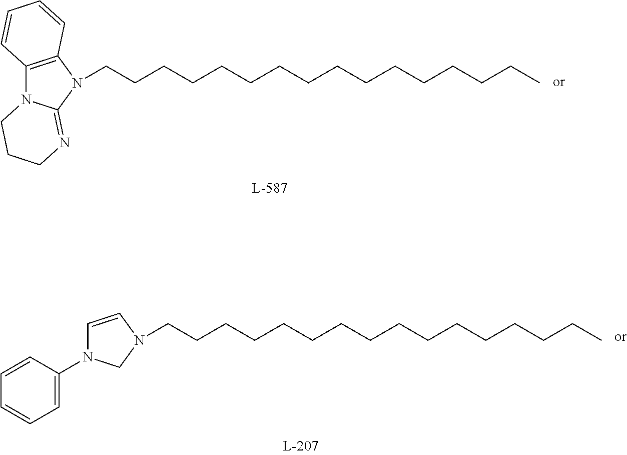

These two compounds are similar in structure to a previously known eEF2K inhibitor known as NH-125 (Arora et al., Mol. Pharmacol., 66(3), 460-467 (2004)). All three of these compounds also bear remarkable resemblance to springosine-1-phosphate, which is a known radioprotector. Sphingosine-1-phosphate was found to inhibit eEF2K activity in vitro, which suggests that eEF2 kinase mediates the radioprotective effects of sphingosine-1-phosphate in vivo.

The foregoing compounds are similar in that they contain a 16 carbon aliphatic chain with a positively charged head group. The compounds structurally resemble sphingosine-1-phosphate, which was tested and also found to be an inhibitor of eEF2 kinase. Again, all of these 16 carbon compounds appear to be structurally similar to previously identified specific inhibitor of eEF2 kinase, NH-125 (Arora, et al., Mol. Pharmacol., 66(3), 460-467). The 16 carbon compounds of this configuration interfere with substrate binding and appear to bind to a C-terminal substrate binding domain of the eEF2 kinase.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

We claim:

1. A method for inhibiting the build-up of amyloid plaques in the brain of a patient with at least one risk factor for or a diagnosis of Alzheimer's Disease, comprising administering to the patient an amount of one or more compounds effective to inhibit the phosphorylative activity of eEF2K, thereby inhibiting amyloid plaque deposition.

2. The method of claim 1 wherein the eEF2K inhibitor is a competitive or noncompetitive inhibitor.

3. The method of claim 1 wherein the eEF2K inhibitor is selected from the group consisting of 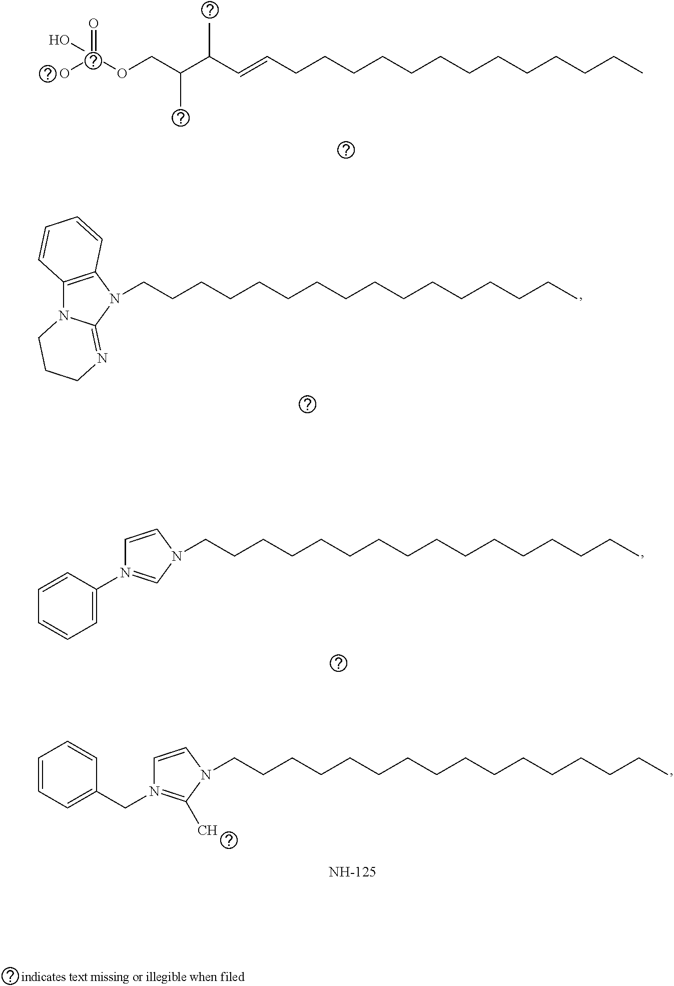
and combinations thereof.

4. The method of claim 1 wherein the eEF2K inhibitors are selected from the group consisting of 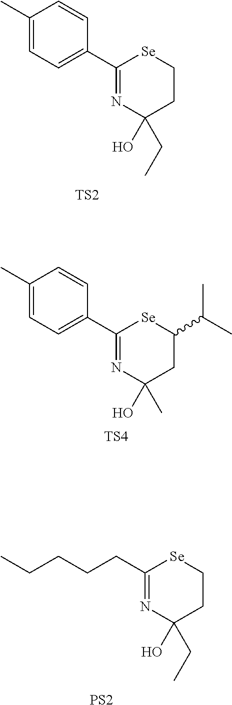
and combinations thereof.

5. The method of claim 1 wherein the eEF2K inhibitors are comprised of chalcone.

6. The method of claim 1 wherein said eEF2K inhibitors are administered in a chronic dose.

7. The method of claim 1 wherein said eEF2K inhibitors are administered orally or intravenously.

8. The method of claim 1, wherein said patient is diagnosed with Alzheimer's Disease.

9. The method of claim 1, wherein said patient has a family history of Alzheimer's Disease.

10. The method of claim 1, wherein said patient has a genetic marker for Alzheimer's Disease.

11. The method of claim 1, wherein said patient suffered a head injury predisposing the patient to Alzheimer's Disease.

12. The method of claim 1, wherein said patient has two or more risk factors for Alzheimer's Disease.

13. The method of claim 1, wherein said eEF2K inhibitor is administered with one or more other agents for treating Alzheimer's disease.

14. The method of claim 13, wherein said other agent(s) are not eEF2K inhibitors.

15. The method of claim 14, wherein the other agent(s) are selected from the group consisting of donepezil, rivastigmine, galantamine, memantine, riluzole and tacrine.
